SEQUENCE LISTING (35 sequences)

SEQ ID NO: 1 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 2 - DNA, 554 bp, synthetic construct
  source 1..554: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 3 - DNA, 393 bp, synthetic construct
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 4 - DNA, 393 bp, synthetic construct
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 5 - DNA, 723 bp, synthetic construct
  source 1..723: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 6 - DNA, 1676 bp, synthetic construct
  source 1..1676: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 7 - DNA, 95 bp, synthetic construct
  source 1..95: mol_type = other DNA
taaaatgggcaaacattgcaagcagcaaacagcaaacacacagccctccctgcctgctgaccttggagctggggcagaggtcagagacctctctg

SEQ ID NO: 8 - DNA, 347 bp, synthetic construct
  source 1..347: mol_type = other DNA
ctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagggagcgg

SEQ ID NO: 9 - DNA, 433 bp, synthetic construct
  source 1..433: mol_type = other DNA
ccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 10 - DNA, 1283 bp, synthetic construct
  source 1..1283: mol_type = other DNA
taaaatgggcaaacattgcaagcagcaaacagcaaacacacagccctccctgcctgctgaccttggagctggggcagaggtcagagacctctctgagaggggtacccgggctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagggagcggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 11 - DNA, 826 bp, synthetic construct
  source 1..826: mol_type = other DNA
ccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 12 - DNA, 351 bp, synthetic construct
  source 1..351: mol_type = other DNA
aaccattgtgggcagccagcctggcgggagaggctagggcacgaagcgccgcccagttaccatggtaacactccctcgtcccaaccccgtcccagtttactggcccaaggtaaggccgggagccgtactccagggcgactgggaggatccactccctgccgcggctctccctccagtggactgccttcccgcctgcctggcggtgggagggcgcggcagagggacgtcacatccgggcgggttggtgagttccggtatttcagggcgtagcaggcggaagtaagggtgagaggaggctgcaacgccgagcggaggaggcaggaaccggagcgcgagcagtagctgggtggg

SEQ ID NO: 13 - DNA, 346 bp, synthetic construct
  source 1..346: mol_type = other DNA
ctcccctcttcctcttcttttaaaacatccatagagctgcccagaatgagtggaaagtttcagggtcagggagaacctcttccctcccccactttccattgtctaaaatcagcctcagctgttgctagatattttttttgctggtgatgtcagttcatcaaagcgtctcaccctgtgttcccccaggagaacaatgatggggtggagggagctggaaagaccatattaggaacaaaaggtggagctatgagcacagataaagactcaagtctggggacctcctggtcactcaggcagcagccccttctttcttgccccagtctccagttctccagtgttcacaggt

SEQ ID NO: 14 - DNA, 330 bp, synthetic construct
  source 1..330: mol_type = other DNA
ggcactggggtgtgtctccttaggacacccctgcccattactctctccttctccaaatggggagcatggctggggctccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggaga

SEQ ID NO: 15 - DNA, 1579 bp, synthetic construct
  source 1..1579: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 16 - skipped

SEQ ID NO: 17 - DNA, 100 bp, synthetic construct
  source 1..100: mol_type = other DNA
aggttaatttttaaaaagcagtcaaaagtccaagtggcccttggcagcatttactctctctgtttgctctggttaataatctcaggagcacaaacattcc

SEQ ID NO: 18 - DNA, 200 bp, synthetic construct
  source 1..200: mol_type = other DNA
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 19 - DNA, 321 bp, synthetic construct; identical to SEQ ID NO: 1
  source 1..321: mol_type = other DNA

SEQ ID NO: 20 - DNA, 554 bp, synthetic construct; identical to SEQ ID NO: 2
  source 1..554: mol_type = other DNA

SEQ ID NO: 21 - DNA, 460 bp, synthetic construct
  source 1..460: mol_type = other DNA
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 22 - DNA, 723 bp, synthetic construct; identical to SEQ ID NO: 5
  source 1..723: mol_type = other DNA

SEQ ID NO: 23 - DNA, 144 bp, synthetic construct
  source 1..144: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 24 - DNA, 288 bp, synthetic construct
  source 1..288: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 25 - DNA, 432 bp, synthetic construct
  source 1..432: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 26 - DNA, 199 bp, synthetic construct
  source 1..199: mol_type = other DNA
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 27 - DNA, 433 bp, synthetic construct; identical to SEQ ID NO: 9
  source 1..433: mol_type = other DNA

SEQ ID NO: 28 - DNA, 328 bp, synthetic construct
  source 1..328: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 29 - DNA, 174 bp, synthetic construct
  source 1..174: mol_type = other DNA
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaag

SEQ ID NO: 30 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 31 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 32 - DNA, 97 bp, synthetic construct
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 33 - DNA, 55 bp, synthetic construct
  source 1..55: mol_type = other DNA
ataaaggaaatttattttcattgcaatagtgtgttggaattttttgtgtctctca

SEQ ID NO: 34 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 35 - protein, 25 aa, synthetic construct
RKRRGSGEGRGSLLTCGDVEENPGP

(19) United States
(12) Patent Application Publication
(10) Pub. No.: US 2025/0025574 A1
(43) Pub. Date: Jan. 23, 2025

(54) ENGINEERED NUCLEIC ACID REGULATORY ELEMENTS AND METHODS AND USES THEREOF

(71) Applicant: REGENXBIO INC., Rockville, MD (US)

(72) Inventors: Randolph Qian, Gaithersburg, MD (US); Chunping Qiao, Chapel Hill, NC (US); Devin McDougald, Boston, MA (US)

(21) Appl. No.: 18/704,628

(22) PCT Filed: Oct. 28, 2022
(86) PCT No.: PCT/US2022/078914

§ 371 Date: Apr. 25, 2024

Related U.S. Application Data
(60) Provisional application No. 63/338,438, filed on May 4, 2022.

(30) Foreign Application Priority Data
Oct. 28, 2021 (WO) PCT/US21/57155

(51) Int. Cl. A61K 48/00; C12N 15/86

(52) U.S. Cl. CPC A61K 48/0058; C12N 15/86; C12N 2750/14143; C12N 2750/14145; C12N 2750/14152; C12N 2800/22

(57) ABSTRACT

The present invention relates to nucleic acid expression cassettes that are engineered to enhance gene expression. Vectors and methods employing the expression cassettes containing novel chimeric regulatory elements are provided. The invention is particularly useful for delivery of transgenes to target cells and confers desirable properties for liver-directed and muscle-gene therapy. Moreover, the invention relates to gene therapy methods of delivery of therapeutics for treating various disorders.

### 1. SEQUENCE LISTING

The contents of the electronic sequence listing submitted herewith as file 38013_0029P1.xml; Size: 45,056 bytes; and Date of Creation: Oct. 24, 2022, is herein incorporated by reference in its entirety.

### 2. FIELD OF THE INVENTION

The present invention relates to nucleic acid regulatory elements engineered to enhance gene expression, methods of employing the regulatory elements and uses thereof. Use of the engineered regulatory elements upstream of a transgene delivered to target cells confers desirable properties, and, in some cases, confers desirable properties for gene therapy. In particular, the invention provides nucleic acid regulatory elements operably linked to a heterologous gene (transgene) inserted into an expression cassette, such that the regulatory elements drive expression of the transgene in specific cells. As such, the invention also provides a method to target tissues, in particular, administering nucleic acids comprising expression cassettes comprising the engineered regulatory elements which improve expression of the transgene in liver and/or muscle, as well as to deliver therapeutics systemically for the treatment of various disorders.

### 3. BACKGROUND

The use of regulatory elements to drive gene expression is highly complex. Both naturally occurring and synthetic regulatory elements, such as enhancers and promoters, have been reported in the art. It is not known whether multiple elements engineered for heterologous gene expression will produce various aberrant, unstable and/or competing transcripts in a given tissue environment.

Gene expression vectors that are highly productive and stable may be suitable for gene therapy. Transgenes delivered with AAV or other viral vectors aim to provide long-term gene expression and thus may boost systemic expression levels or serum half-life of a biotherapeutic transgene. As such, improved gene expression systems for gene therapy would greatly benefit patients compared to direct injection of a biologic drug, such as in enzyme replacement therapy. Although AAV capsid proteins that carry genomic DNA can confer a particular tissue tropism to deliver DNA into target cells, it is desirable to express greater amounts of the gene of interest in liver, due to its low immunogenicity (Pastore, et al. Human Gene Therapy Vol. 10, No. 11, July 1999 online ahead of print).

Thus, liver and muscle expression of a biotherapeutic would be desirable to elevate serum levels and systemic delivery of the protein. There remains a need for tissue-targeted gene expression and vectors that are highly productive in liver and/or skeletal muscle.

### 4. SUMMARY OF THE INVENTION

Provided are recombinant expression cassettes comprising a composite nucleic acid regulatory element for enhancing or directing gene expression in the liver and in skeletal muscle. The regulatory element is a composite comprising at least two enhancers and at least two promoters, operably linked to a transgene. In some embodiments, the composite nucleic acid regulatory element comprises two promoters arranged in tandem where the downstream or 3′ promoter is start codon-modified (for example, deleted for the start codon (ΔATG)).

Provided are recombinant expression cassettes comprising a composite nucleic acid regulatory element which comprises two promoters arranged in tandem, where the promoters are a muscle-specific promoter and a liver-specific promoter, including promoters in Table 1. In embodiments, the promoters are the hAAT promoter (for example, SEQ ID NO: 3 or 4) and the CK promoter (for example, SEQ ID NO: 9). In one embodiment, the hAAT promoter is the downstream promoter in the arrangement and is start-codon modified (that is deleted for the start codon or ΔATG) (for example, SEQ ID NO: 4, and in combination with the CK promoter, SEQ ID NO: 11), wherein the composite nucleic acid regulatory element is operably linked to a transgene. In some embodiments, the composite nucleic acid regulatory element comprises a muscle enhancer (e.g., Mus022 (SEQ ID NO: 8), Mus007 (SEQ ID NO: 12), Mus011 (SEQ ID NO:13) or Mus035 (SEQ ID NO:14)), a muscle specific promoter and a start-codon modified (ΔATG) hAAT promoter. In some embodiments, the CK promoter is the downstream promoter and is start-codon modified (ΔATG), wherein the nucleic acid regulatory element is operably linked to a transgene. The composite nucleic acid regulatory element further comprises one or more enhancer elements. The enhancer elements may also be liver-specific and/or muscle specific. In embodiments, the composite nucleic acid regulatory element comprises one or two muscle specific cis regulatory element (CRE) or Mus CRE. In embodiments the Mus CRE is Mus022 (SEQ ID NO: 8), Mus007 (SEQ ID NO: 12), Mus011 (SEQ ID NO: 13), or Mus035 (SEQ ID NO: 14) (see Table 1). In embodiments, the composite nucleic acid regulatory element also comprise an ApoE enhancer, including a synthetic ApoE enhancer (SEQ ID NO: 7; Table 1) which may be either 5′ or 3′ of the Mus CRE (and 5′ of the promoter sequences).

In embodiments, the promoter is a LMTP24 promoter which is a tandem liver/muscle specific enhancer promoter. The LMTP24 promoter is comprised of (i) synthetic ApoE enhancer region (SEQ ID NO: 7). (ii) a muscle enhancer region (for example, Mus022, SEQ ID NO: 8)), (iii) a CK promoter (SEQ ID NO: 9), and (IV) a hAAT promoter (ΔATG) (SEQ ID NO: 4). Optionally, an intron sequence (see, e.g., intron sequences in Table 2) is between the composite regulatory element and the transgene. The LMTP24 promoter may have a nucleotide sequence of SEQ ID NO: 10. See also FIG. 1 for arrangement of elements.

In some embodiments, provided are expression cassettes comprising the composite nucleic acid regulatory element, including LMTP24, which is operably linked to a transgene. The transgene may be any one of the genes or nucleic acids encoding the therapeutic proteins listed in, but not limited to, Tables 4A-4D. In certain embodiments, the transgene encodes a therapeutic antibody, either having full length heavy and light chains, or an antigen binding fragment, such as a Fab fragment or an scFv. In embodiments, the expression cassette is flanked by AAV ITR sequences and may be within a cis plasmid construct for AAV particle production or an artificial genome within an AAV capsid.

In some embodiments, the vectors comprise a transgene operably linked to a composite nucleic acid regulatory element comprising or consisting of a nucleic acid sequence which has a muscle CRE (for example, see Table 1) 5′ of a tandem liver specific promoter and a muscle-specific promoter. In embodiments, the liver specific promoter is hAAT promoter and the muscle specific promoter is a CK promoter. In embodiments, the composite nucleic acid regulatory element comprises a further enhancer element, including an ApoE enhancer element, such as a synthetic ApoE enhancer element (see, e.g., Table 1). In embodiments, the composite nucleic acid regulatory element is LMTP24, including an element with a nucleotide sequence of SEQ ID NO: 10, wherein the transgene under its control is expressed in both the liver and the muscle, including skeletal muscle. In certain embodiments, the transgene under control of the regulatory element, is also expressed in cardiac muscle or heart tissue or has reduced expression in cardiac muscle or heart tissue when compared to expression in muscle and/or liver tissue.

Also provided are methods for enhancing expression of a transgene in one or more target cells or target tissues, including liver and/or muscle, comprising delivery of viral vectors comprising nucleic acid expression cassettes comprising a 5′ to 3′ arrangement of a) more than one, for example, muscle enhancer regions, such muscle CRE elements, including Mus022 and other muscle CRE elements in Table 1 b) two or more promoter sequences, for example a muscle specific promoter and a liver specific promoter, wherein at least one liver-specific promoter comprises a modified start codon (ΔATG) or at least one muscle specific promoter comprises a modified start codon (ΔATG) (including where the promoter with the modified start codon is the 3′ most element or, alternatively, is 5′ of the other promoter element), and c) a transgene. Optionally, the composite nucleic acid regulatory element comprises an ApoE enhancer, such as a synthetic ApoE enhancer (SEQ ID NO: 7), and an intron sequence (see, e.g., intron sequences in Table 2) may be situated between the composite nucleic acid regulatory element and the transgene.

In some embodiments, provided are viral vectors incorporating the engineered expression cassettes described herein, including rAAVs.

In another aspect, methods of treatment by delivery of rAAVs comprising the nucleic acid expression cassettes described herein are also provided. In embodiments, provide is a method for treating a disease or disorder in a subject in need thereof comprising the administration of recombinant AAV particles comprising an expression cassette having a composite regulatory sequence comprising a muscle CRE, such as Mus022, a muscle specific promoter, including a CK promoter, and a liver specific promoter, including an hAAT promoter (which may have a modified start codon ΔATG) and optionally 5′ or 3′ of the muscle CRE an ApoE enhancer operably linked to a transgene which expresses a therapeutic product for treatment of the disease or disorder. In embodiments, the composite regulatory sequence is LMTP24.

Also provided are methods of producing recombinant AAV vectors comprising an expression cassette with AAV comprising a composite nucleic acid regulatory element described herein operably linked to a transgene by culturing a host cell comprising an artificial genome flanked by AAV ITRs and comprising the nucleic acid regulatory element, including LMTP24, operably linked to the transgene and a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans; sufficient adenovirus helper functions to permit replication and packaging of the artificial genome by the AAV capsid protein; and recovering recombinant AAV encapsidating the artificial genome from the cell culture. Host cells for production of the recombinant AAV described herein are also provided. Also provided are host cells comprising a plasmid vector encoding the AAV recombinant genome comprising expression cassettes comprising the regulatory elements operably linked to a transgene and flanked by AAV ITR sequences.

### EMBODIMENTS

1. A recombinant expression cassette comprising a composite nucleic acid regulatory element comprising a) Muscle cis regulatory element (CRE) and b) at least two promoters arranged in tandem wherein at least one promoter the is a muscle specific and one promoter is hAAT, wherein the hAAT is start-codon deleted (ΔATG) (SEQ ID NO: 4), operably linked to a transgene.
2. A recombinant expression cassette comprising a composite nucleic acid regulatory element comprising a) Muscle cis regulatory element (CRE) and b) a muscle specific promoter, operably linked to a transgene.
3. The recombinant expression cassette of embodiment 1 or 2 wherein the muscle specific promoter is a CK promoter (SEQ ID NO: 9).
4. The recombinant expression cassette of embodiment 2 wherein the muscle specific promoter is from Table 1.
5. The recombinant expression cassette of any one of embodiments 1 to 4 wherein the muscle CRE is Mus022, Mus077, Mus011 or Mus035.
6. The recombinant expression cassette of embodiment 5 wherein the muscle CRE is Mus022.
7. The recombinant expression cassette of any one of embodiments 1 to 6 further comprising one or two copies of synthetic ApoE (SEQ ID NO: 7) either 5′ of or 3′ of the muscle CRE.
8. The recombinant expression cassette of any one of embodiments 1 to 7, wherein the nucleic acid regulatory element is LMTP24 (SEQ ID NO. 10) of Table 1.
9. The recombinant expression cassette of any one of embodiments 1 to 8 which further comprises an intron sequence between the nucleic acid regulatory element and the transgene.
10. The expression cassette of any one of embodiments 1 to 9, where the transgene is a gene or nucleic acid encoding any of the therapeutics listed in Tables 4A-4D.
11. The expression cassette of any one of embodiments 1 to 10, wherein the transgene encodes a therapeutic antibody, or antigen binding fragment thereof.
12. A vector comprising the expression cassette of any one of embodiments 1 to 11.
13. The vector of embodiment 12, further comprising AAV ITRs flanking the expression cassette.
14. The vector of embodiments 12 or 13 wherein the cassette is suitable for packaging in an AAV capsid.
15. The vector of any one of embodiments 12 to 14 comprising an artificial genome comprising (1) AAV inverted terminal repeats (ITRs) flanking the expression cassette; (2) an expression cassette comprising (a) a composite nucleic acid regulatory control element comprising a nucleic acid sequence of SEQ ID NO: 10; b) a poly A signal, c) optionally an intron; and d) a transgene coding for one or more RNA or protein products to which the composite nucleic acid regulatory element is operably linked.
16. An rAAV particle comprising the vector of any one of embodiments 12 to 15, and a capsid protein from an AAV capsid serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV. Anc80, AAV.Anc80L65, AAV.7m8, AAV.PHP.B. AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10. AAV.HSC11, AAV.HSC12, AAV.HSC13, AAV.HSC14, AAV.HSC15, or AAV.HSC16, or a derivative, modification, or pseudotype thereof.
17. A method for enhancing expression of a transgene in a subject, said method comprising delivery of viral vectors comprising a nucleic acid expression cassette having a 5′ to 3′ arrangement of a) Mus CRE sequence, b) at least one muscle-specific promoter and at least one liver-specific promoter, wherein the 3′ promoter comprises a deleted start codon, c) a transgene; and d) a polyadenylation signal sequence.
18. The method of embodiment 17 wherein the muscle specific promoter is CK promoter (SEQ ID NO: 9) and the liver-specific promoter is an hAAT (ΔATG) promoter and/or the Mus CRE sequence is a Mus022 (SEQ ID NO: 8), Mus007 (SEQ ID NO: 12), Mus011 (SEQ ID NO: 13), or Mus035 (SEQ ID NO: 14) sequence.
19. The method of embodiments 17 or 18 further comprising a synthetic ApoE enhancer either 5′ of or 3′ of the Mus CRE sequence.
20. The method of any one of embodiments 17 to 19 wherein the nucleic acid expression cassette comprises a nucleic acid regulatory element of LMTP24 (SEQ ID NO: 10).
21. The method of any one of embodiments 17 to 20, wherein the viral vector is administered intravenously or intramuscularly.
22. The method of any one of embodiments 17 to 21, wherein transgene expression is enhanced in the circulation or systemically.
23. The method of any one of embodiments 17 to 22, wherein the transgene expression is enhanced in the liver or skeletal muscle.
24. The method of any one of the embodiments 17 to 23, wherein the transgene expression in enhanced relative to expression from a comparable viral vector having only a muscle specific promoter, or only a liver specific promoter or an LMTP6 (SEQ ID NO: 15) promoter operably linked to the transgene.
25. A method of treatment comprising administering rAAVs comprising the expression cassettes of any one of embodiments 1-11, vectors of any one of embodiments 12 to 14, or rAAV of embodiment 15.
26. A method for treating a disease or disorder in a subject in need thereof comprising administering recombinant AAV particles comprising an expression cassette having a synthetic ApoE enhancer sequence (SEQ ID NO: 7) and/or a Mus CRE sequence upstream of a muscle-specific promoter and a liver-specific promoter, wherein at least the muscle specific promoter or the liver-specific promoter comprises a deleted start codon (ΔATG), operably linked to a transgene.
27. The method of embodiment 26 wherein the muscle specific promoter is the CK promoter (SEQ ID NO: 9) and the liver-specific promoter is hAAT (SEQ ID NO: 3 or 4) and/or the Mus CRE sequence is a Mus022 (SEQ ID NO: 8), Mus007 (SEQ ID NO: 12), Mus011 (SEQ ID NO: 13), or Mus035 (SEQ ID NO: 14) sequence.
28. The method of embodiment 27 wherein the hAAT promoter is 3′ of the muscle specific promoter and has a deleted start codon.
29. The method of any one of the embodiments 17 to 28, wherein the expression cassette comprises a composite regulatory sequence having the nucleic acid sequence of SEQ ID NO: 10.
30. The method of any one of embodiments 17 to 29, wherein the transgene is selected from Tables 4A-4D.
31. The method of any one of embodiments 17 to 30, wherein the transgene encodes a therapeutic antibody, or antigen binding fragment thereof.
32. The method of any one of embodiments 17 to 31, wherein the rAAV is administered intravenously or intramuscularly.
33. The method of any one of the embodiments 26 to 32, wherein the transgene expression in enhanced relative to expression from a comparable viral vector having only a muscle specific promoter, or only a liver specific promoter or an LMTP6 promoter (SEQ ID NO: 15) operably linked to the transgene.
34. A method of producing recombinant AAVs comprising:
culturing a host cell containing:
   an artificial genome comprising a cis expression cassette flanked by AAV ITRs, wherein the cis expression cassette comprises a composite nucleic acid regulatory element comprising a) a synthetic ApoE Enhancer and a Mus022 enhancer, and b) at least two promoters arranged in tandem wherein at least one promoter is a CK promoter and at least one promoter is a hAAT, wherein the hAAT is start-codon deleted (ΔATG), operably linked to a transgene coding for one or more RNA or protein products;
   a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
   sufficient adenovirus helper functions to permit replication and packaging of the artificial genome by the AAV capsid protein; and
recovering recombinant AAV encapsidating the artificial genome from the cell culture.
35. The method of embodiment 34, wherein the composite nucleic acid regulatory element is LMTP24.
36. A host cell comprising (i) a plasmid comprising a cis expression cassette flanked by AAV ITRs, wherein the cis expression cassette comprises a composite nucleic acid regulatory element comprising a) a synthetic ApoE Enhancer and a Mus022 enhancer, and b) at least two promoters arranged in tandem wherein at least one promoter is a CK promoter and at least one promoter is a hAAT, wherein the hAAT is start-codon deleted (ΔATG), operably linked to a transgene coding for one or more RNA or protein products.
37. The host cell of embodiment 36, wherein the composite nucleic acid regulatory element is LMTP24 (SEQ ID NO: 10).
38. A pharmaceutical composition for enhancing expression of a transgene in a subject, said pharmaceutical composition comprising viral vectors comprising a nucleic acid expression cassette having a 5′ to 3′ arrangement of a) a Mus CRE sequence, b) at least one muscle-specific promoter and at least one liver-specific promoter, wherein the 3′ promoter comprises a deleted start codon, c) a transgene; and d) a polyadenylation signal sequence.
39. The pharmaceutical composition of embodiment 38 wherein the muscle specific promoter is CK promoter (SEQ ID NO: 9) and the liver-specific promoter is an hAAT (ΔATG) promoter and/or the Mus CRE sequence is a Mus022 (SEQ ID NO: 8), Mus007 (SEQ ID NO: 12), Mus011 (SEQ ID NO: 13), or Mus035 (SEQ ID NO: 14) sequence.
40. The pharmaceutical composition of embodiments 38 or 39 further comprising a synthetic ApoE enhancer either 5′ of or 3′ of the Mus022 sequence.
41. The pharmaceutical composition of any one of embodiments 38 to 40 wherein the nucleic acid expression cassette comprises a nucleic acid regulatory element of LMTP24 (SEQ ID NO: 10).
42. The pharmaceutical composition of any one of embodiments 38 to 41, formulated for intravenous or intramuscular administration.
43. The pharmaceutical composition of any one of embodiments 38 to 42, wherein transgene expression is enhanced in the circulation or systemically.
44. The pharmaceutical composition of any one of embodiments 38 to 43, wherein the transgene expression is enhanced in the liver or skeletal muscle.
45. A pharmaceutical composition for use in treating a disease or disorder in a subject in need thereof comprising rAAVs comprising the expression cassettes of any one of embodiments 1-12, vectors of any one of embodiments 13 to 16, or rAAV of embodiment 17.
46. A pharmaceutical composition for use in treating a disease or disorder in a subject in need thereof comprising recombinant AAV particles comprising an expression cassette having a synthetic ApoE enhancer sequence (SEQ ID NO: 7) and/or a Mus CRE upstream of a muscle-specific promoter and a liver-specific promoter, wherein at least the muscle specific promoter or the liver-specific promoter comprises a deleted start codon (ΔATG), operably linked to a transgene.
47. The pharmaceutical composition of embodiment 46 wherein the muscle specific promoter is the CK promoter (SEQ ID NO: 9) and the liver-specific promoter is hAAT (SEQ ID NO: 3 or 4) and/or the Mus CRE sequence is a Mus022 (SEQ ID NO: 8), Mus007 (SEQ ID NO: 12), Mus011 (SEQ ID NO: 13), or Mus035 (SEQ ID NO: 14) sequence.
48. The pharmaceutical composition of embodiment 47 wherein the hAAT promoter is 3′ of the muscle specific promoter and has a deleted start codon.
49. The pharmaceutical composition of any one of the embodiments 38 to 48, wherein the expression cassette comprises a composite regulatory sequence having the nucleic acid sequence of SEQ ID NO: 10.
50. The pharmaceutical composition of any one of embodiments 38 to 49, wherein the transgene is selected from Tables 4A-4D.
51. The pharmaceutical composition of any one of embodiments 38 to 50, wherein the transgene encodes a therapeutic antibody, or antigen binding fragment thereof.
52. The pharmaceutical composition of any one of embodiments 38 to 51, formulated for intravenous or intramuscular injection.
33. The pharmaceutical composition of any one of the embodiments 38 to 52, wherein the transgene expression in enhanced relative to expression from a comparable viral vector having only a muscle specific promoter, or only a liver specific promoter or an LMTP6 promoter operably linked to the transgene.

The invention is illustrated by way of examples infra describing the construction and function of gene cassettes engineered with composite regulatory elements designed on the basis of several liver-specific enhancers and promoters, in tandem with muscle specific enhancers and promoters, whereas the downstream elements are modified at their translation start sites.

### 5. BRIEF DESCRIPTION OF THE FIGURES

FIG. 1 Depiction of an AAV genome cassette, arranged 5′ to 3′: 5′-ITR, muscle CRE (Mus CRE), CK promoter, hAAT (ΔATG) promoter, optional intron, gene of interest, poly adenylation (polyA) sequence, 5′-ITR. The regulatory elements comprising the muscle-specific enhancer/CRE, CK promoter and hAAT (ΔATG) promoter create a muscle-liver tandem promoter (LMTP).

FIGS. 2A-2C: FIG. 2A: Depiction of a reporter gene cassette, arranged 5′ to 3″: 5′-ITR, muscle CRE (Mus CRE), CK promoter, intron, eGFP transgene, barcode sequence, poly adenylation (polyA) sequence, 3′-ITR. FIG. 2B: Representative micrographs for each CRE candidate cassette as transfected into differentiated C2C12 cells and exhibiting eGFP expression (fluorescence). FIG. 2C: Data graph depicting fold-change of CRE activity for each plasmid normalized to a control plasmid (CK promoter with no upstream CRE).

FIG. 3 depicts transgene RNA transcripts per μg RNA in liver, heart and skeletal muscle (Quad) tissue of Cynomolgus monkeys administered AAV particles as indicated.

FIG. 4. depicts serum concentration of transgene expressed antibodies from Cynomolgus monkeys administered the AAV particles as indicated over 92 days from administration.

### 6. DETAILED DESCRIPTION

Provided are unique combinations of promoter and enhancer sequences in expression cassettes suitable for improvement of transgene expression in one or more target cells or target tissues while maintaining or conferring tissue specificity. Provided are vectors, such as viral vectors, incorporating the engineered expression cassettes described herein, including rAAVs, for use in therapy, and methods and host cells for producing same. The novel regulatory element nucleic acids were generated to improve transgene expression from tandem promoters (i.e. two promoter sequences driving expression of the same transgene) by depleting the 3′ promoter sequence of potential ‘ATG’ initiation sites. This approach was employed to improve transgene expression from tandem tissue-specific promoter cassettes (such as those targeting the liver) as well as promoter cassettes to achieve dual expression in at least two separate tissue populations (such as liver and skeletal muscle). Ultimately, these designs may improve the therapeutic efficacy of gene transfer by providing more robust levels of transgene expression, improved stability/persistence, and induction of immune tolerance to the transgene product. Provided are composite regulatory elements which promote expression in skeletal muscle and liver cells while having minimal expression in heart or cardiac muscle tissue.

### 6.1. Definitions

The term “regulatory element” or “nucleic acid regulatory element” are non-coding nucleic acid sequences that control the transcription of neighboring genes. Cis regulatory elements typically regulate gene transcription by binding to transcription factors. This includes “composite nucleic acid regulatory elements” comprising more than one enhancer or promoter elements as described herein.

The term “expression cassette” or “nucleic acid expression cassette” refers to nucleic acid molecules that include one or more transcriptional control elements including, but not limited to promoters, enhancers and/or regulatory elements, introns and polyadenylation sequences. The enhancers and promoters typically function to direct (trans) gene expression in one or more desired cell types, tissues or organs.

The term “operably linked” and “operably linked to” refers to nucleic acid sequences being linked and typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked and still be functional while not directly contiguous with a downstream promoter and transgene.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into or modification of the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

The terms “subject”, “host”, and “patient” are used interchangeably. As used herein, a subject is preferably a mammal such as a non-primate (e.g., cows, pigs, horses, cats, dogs, rats etc.) or a primate (e.g., monkey and human), most preferably a human.

The terms “therapeutic agent” or “biotherapeutic agent” refer to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The phrase “liver-specific” or “liver-directed” refers to nucleic acid elements that have adapted their activity in liver (hepatic) cells or tissue due to the interaction of such elements with the intracellular environment of the hepatic cells. The liver acts as a bioreactor or “depot” for the body in the context of a gene therapy delivered to the liver tissue and a gene cassette enhanced for expression in the liver will produce the biotherapeutic (translated protein) that is secreted into the circulation. As such, the biotherapeutic agent is delivered systemically to the subject by way of liver expression. Without being bound by any one theory, liver production of a biotherapeutic agent (such as produced by the delivered transgene) can provide immunotolerance to the agent such that endogenous T cells of the subject producing the protein will recognize the protein as self-protein, and not induce an innate immune response.

The phrase “muscle-specific” or “muscle-directed” refers to nucleic acid elements that have adapted their activity in muscle cells or tissue due to the interaction of such elements with the intracellular environment of the muscle cells. Muscle cells include skeletal muscle as well as cardiac muscle. Secretion of transgene product into the muscle, and/or bloodstream may also be enhanced following various routes of administration, such as intravenous or intramuscular administration, due to intramuscular expression where muscle-specific promoters are present. Various therapeutics benefit from muscle-specific expression of the transgene, or from both muscle-specific and liver-specific expression of the transgene. Muscle production of a biotherapeutic agent (such as produced by the delivered transgene) may provide also provide the host with increased immunotolerance to the agent, as compared to direct injection of an equivalent protein agent to the host.

### 6.2. Regulatory Elements

One aspect relates to nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

Provided are arrangements of combinations of nucleic acid regulatory elements that promote transgene expression in liver and muscle (including skeletal muscle) tissue. In particular, certain elements are arranged with one or more copies of the individual enhancer and promoter elements arranged in tandem and operably linked to a transgene to promote expression, particularly tissue specific expression. Exemplary nucleotide sequences of the individual promoter and enhancer elements are provided in Table 1. Also provided in Table 1 are exemplary composite nucleic acid regulatory elements comprising the individual tandem promoter and enhancer elements. In certain embodiments the downstream promoter is an hAAT promoter (for example, SEQ ID NO: 3) (in certain embodiments the hAAT promoter is an hAAT (ΔATG) promoter (for example, SEQ ID NO: 4)) and the other promoter is a muscle specific promoter, including a CK promoter (for example, SEQ ID NO: 9).

Accordingly, with respect to liver and muscle specific expression, provided are nucleic acid regulatory elements that comprise or consist of promoters and other nucleic acid elements, such as enhancers. In embodiments, the enhancers enhance muscle specific expression, such muscle CREs, including Mus022 (SEQ ID NO: 8), and also including, Mus007 (SEQ ID NO: 12), Mus011 (SEQ ID NO: 13) and Mus035 (SEQ ID NO: 14). In other embodiments, the composite nucleic acid regulatory element comprises an enhancer (either 5′ or 3′ of the Mus CRE) that promotes liver expression, such as ApoE enhancers (for example the synthetic ApoE enhancer of SEQ ID NO: 7). These elements may be present as single copies or with two or more copies in tandem.

The recombinant expression cassettes provided herein comprise i) a composite nucleic acid regulatory element comprising a) a muscle specific enhancer region, for example, a Mus CRE, including Mus022 (SEQ ID NO: 8), Mus007 (SEQ ID NO: 12), Mus011 (SEQ ID NO: 13) or Mus035 (SEQ ID NO: 14), b) a muscle-specific promoter, including a CK promoter (for example, SEQ ID NO: 9), and c) an hAAT promoter, including which is start-codon modified (ΔATG) (for example, SEQ ID NO: 4) (where in certain embodiments the hAAT promoter is the downstream or 3′ promoter) and optionally 5′ or 3′ of the muscle specific enhancer region, a liver specific enhancer, such as an ApoE enhancer, and ii) a transgene, to which the composite nucleic acid regulatory element is operably linked, and other regulatory elements, such as a polyadenylation signal. In some embodiments, the composite nucleic acid regulatory element comprises or consists of LMTP24 (SEQ ID NO: 10) of Table 1. In some embodiments, the composite nucleic acid regulatory element is operably linked to a transgene. The transgene may be any one of the genes or nucleic acids encoding the therapeutic proteins listed in, but not limited to, Tables 4A-4D. The transgene may also encode a therapeutic antibody, including a full length antibody or an antigen binding fragment, such as a Fab fragment. In embodiments, the antigen binding fragment comprises the binding domain of the antibody, for example, comprising the CDR sequences or the VH and VL domains or other portion that binds to the antigen. Alternatively, the transgene may encode a nucleic acid therapeutic.

Also provided are nucleic acid regulatory elements which comprise a Muscle CRE (including Mus022, Mus007, Mus011, or Mus035) upstream (5′) of a muscle-specific promoter, including CK promoter or any other muscle specific promoter (see Table 1), for example, Spc5-12 which, in embodiments, are operably linked to a transgene.

Provided are composite regulatory elements that enhance gene expression in the liver and skeletal muscle which have 99%, 95%, 90%, 85% or 80% sequence identity with SEQ ID NO: 10 (LMTP24).

In an aspect of the invention, various regulatory elements and combinations of elements were utilized to design and generate nucleic acid expression cassettes, and are listed in Table 1.

TABLE 1
Promoter sequences

| Name | SEQ ID NO: | Nucleic Acid Sequence |
|---|---|---|
| ApoE Hepatic | SEQ ID NO: | aggctcagaggcacacaggagtttctgggctcaccctgcccccttccaa |
| Control Region | 1 | cccctcagttcccatcctccagcagctgtttgtgtgctgcctctgaagt |
| containing | | ccacactgaacaaacttcagcctactcatgtccctaaaatgggcaaaca |
| ApoE | | ttgcaagcagcaaacagcaaacacacagccctccctgcctgctgacctt |
| Enhancer | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggg |
| Tandem (2) | SEQ ID NO: | aggctcagaggcacacaggagtttctgggctcaccctgcccccttccaa |
| ApoE | 2 | cccctcagttcccatcctccagcagctgtttgtgtgctgcctctgaagt |
| Enhancers | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagaaggctcagaggcacac |
| | | aggagtttctgggctcaccctgcccccttccaacccctcagttcccatc |
| | | ctccagcagctgtttgtgtgctgcctctgaagtccacactgaacaaact |
| | | tcagcctactcatgtccctaaaatgggcaaacattgcaagcagcaaaca |
| | | gcaaacacacagccctccctgcctgctgaccttggagctggggcagagg |
| | | tcagagacctctctg |
| hAAT | SEQ ID NO: | gatcttgctaccagtggaacagccactaaggattctgcagtgagagcag |
| Promoter | 3 | agggccagctaagtggtactctcccagagactgtctgactcacgccacc |
| | | ccctccaccttggacacaggacgctgtggtttctgagccaggtacaatg |
| | | actcctttcggtaagtgcagtggaagctgtacactgcccaggcaaagcg |
| | | tccgggcagcgtaggcgggcgactcagatcccagccagtggacttagcc |
| | | cctgtttgctcctccgataactggggtgaccttggttaatattcaccag |
| | | cagcctcccccgttgcccctctggatccactgcttaaatacggacgagg |
| | | acagggccctgtctcctcagcttcaggcaccaccactgacctgggacag |
| | | t |
| hAAT(ΔATG) | SEQ ID NO: | gatcttgctaccagtggaacagccactaaggattctgcagtgagagcag |
| Promoter | 4 | agggccagctaagtggtactctcccagagactgtctgactcacgccacc |
| | | ccctccaccttggacacaggacgctgtggtttctgagccaggtacagtg |
| | | actcctttcggtaagtgcagtggaagctgtacactgcccaggcaaagcg |
| | | tccgggcagcgtaggcgggcgactcagatcccagccagtggacttagcc |
| | | cctgtttgctcctccgataactggggtgaccttggttaatattcaccag |
| | | cagcctcccccgttgcccctctggatccactgcttaaatacggacgagg |
| | | acagggccctgtctcctcagcttcaggcaccaccactgacctgggacag |
| | | t |
| ApoE.hAAT | SEQ ID NO: | aggctcagaggcacacaggagtttctgggctcaccctgcccccttccaa |
| | 5 | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggggtacccggggatcttgctacca |
| | | gtggaacagccactaaggattctgcagtgagagcagagggccagctaag |
| | | tggtactctcccagagactgtctgactcacgccaccccctccaccttgg |
| | | acacaggacgctgtggtttctgagccaggtacaatgactcctttcggta |
| | | agtgcagtggaagctgtacactgcccaggcaaagcgtccgggcagcgta |
| | | ggcgggcgactcagatcccagccagtggacttagcccctgtttgctcct |
| | | ccgataactggggtgaccttggttaatattcaccagcagcctcccccgt |
| | | tgcccctctggatccactgcttaaatacggacgaggacagggccctgtc |
| | | tcctcagcttcaggcaccaccactgacctgggacagt |
| CAG Promoter | SEQ ID NO: | gacattgattattgactagttattaatagtaatcaattacggggtcatt |
| | 6 | agttcatagcccatatatggagttccgcgttacataacttacggtaaat |
| | | ggcccgcctggctgaccgcccaacgacccccgcccattgacgtcaataa |
| | | tgacgtatgttcccatagtaacgccaatagggactttccattgacgtca |
| | | atgggtggagtatttacggtaaactgcccacttggcagtacatcaagtg |
| | | tatcatatgccaagtacgccccctattgacgtcaatgacggtaaatggc |
| | | ccgcctggcattatgcccagtacatgaccttatgggactttcctacttg |
| | | gcagtacatctacgtattagtcatcgctattaccatggtcgaggtgagc |
| | | cccacgttctgcttcactctccccatctcccccccctccccacccccaa |
| | | ttttgtatttatttattttttaattattttgtgcagcgatgggggcggg |
| | | gggggggggggggcgcgcgccaggcggggcggggcggggcgaggggcgg |
| | | ggcggggcgaggcggagaggtgcggcggcagccaatcagagcggcgcgc |
| | | tccgaaagtttccttttatggcgaggcggcggcggcggcggccctataa |
| | | aaagcgaagcgcgcggcgggcgggagtcgctgcgcgctgccttcgcccc |
| | | gtgccccgctccgccgccgcctcgcgccgcccgccccggctctgactga |
| | | ccgcgttactcccacaggtgagcgggcgggacggcccttctcctccggg |
| | | ctgtaattagcgcttggtttaatgacggcttgtttcttttctgtggctg |
| | | cgtgaaagccttgaggggctccgggagggccctttgtgcggggggagcg |
| | | gctcggggggtgcgtgcgtgtgtgtgtgcgtggggagcgccgcgtgcgg |
| | | ctccgcgctgcccggcggctgtgagcgctgcgggcgcggcgcggggctt |
| | | tgtgcgctccgcagtgtgcgcgaggggagcgcggccgggggcggtgccc |
| | | cgcggtgcggggggggctgcgaggggaacaaaggctgcgtgcggggtgt |
| | | gtgcgtgggggggtgagcagggggtgtgggcgcgtcggtcgggctgcaa |
| | | ccccccctgcacccccctccccgagttgctgagcacggcccggcttcgg |
| | | gtgcggggctccgtacggggcgtggcgcggggctcgccgtgccgggcgg |
| | | ggggtggcggcaggtgggggtgccgggcggggcggggccgcctcgggcc |
| | | ggggagggctcgggggaggggcgcggcggcccccggagcgccggcggct |
| | | gtcgaggcgcggcgagccgcagccattgccttttatggtaatcgtgcga |
| | | gagggcgcagggacttcctttgtcccaaatctgtgcggagccgaaatct |
| | | gggaggcgccgccgcaccccctctagcgggcgcggggcgaagcggtgcg |
| | | gcgccggcaggaaggaaatgggcggggagggccttcgtgcgtcgccgcg |
| | | ccgccgtccccttctccctctccagcctcggggctgtccgcggggggac |
| | | ggctgccttcgggggggacggggcagggcggggttcggcttctggcgtg |
| | | tgaccggcggctctagagcctctgctaaccatgttcatgccttcttctt |
| | | tttcctacag |
| Synthetic | SEQ ID NO: | taaaatgggcaaacattgcaagcagcaaacagcaaacacacagccctcc |
| ApoE | 7 | ctgcctgctgaccttggagctggggcagaggtcagagacctctctg |
| Enhancer | | |
| Mus022 | SEQ ID NO: | ctcccgtttgacccaagcttcctgagctctcctcccattcccctttttg |
| | 8 | gagtcctcctcctctcccagaacccagtaataagtgggctcctccctgg |
| | | cctggacccccgtggtaaccctataaggcgaggcagctgctgtctgagg |
| | | cagggaggggctggtgtgggaggctaagggcagctgctaagtttagggt |
| | | ggctccttctctcttcttagagacaacaggtggctggggcctcagtgcc |
| | | cagaaaagaaaatgtcttagaggtatcggcatgggcctggaggaggggg |
| | | gacagggcagggggaggcatcttcctcaggacatcgggtcctagaggga |
| | | gcgg |
| CK Promoter | SEQ ID NO: | ccctgcatgcgaagatcttcgaacaaggctgtgggggactgagggcagg |
| | 9 | ctgtaacaggcttgggggccagggcttatacgtgcctgggactcccaaa |
| | | gtattactgttccatgttcccggcgaagggccagctgtcccccgccagc |
| | | tagactcagcacttagtttaggaaccagtgagcaagtcagcccttgggg |
| | | cagcccatacaaggccatggggctgggcaagctgcacgcctgggtccgg |
| | | ggtgggcacggtgcccgggcaacgagctgaaagctcatctgctctcagg |
| | | ggcccctccctggggacagcccctcctggctagtcacaccctgtaggct |
| | | cctctatataacccaggggcacaggggctgccctcattctaccaccacc |
| | | tccacagcacagacagacactcaggagccagccagcgtcga |
| LMTP24 | SEQ ID NO: | TAAAATGGGCAAACATTGCAAGCAGCAAACAGCAAACACACAGCCCTCC |
| | 10 | CTGCCTGCTGACCTTGGAGCTGGGGCAGAGGTCAGAGACCTCTCTGAGA |
| | | GGGGTACCCGGGCTCCCGTTTGACCCAAGCTTCCTGAGCTCTCCTCCCA |
| | | TTCCCCTTTTTGGAGTCCTCCTCCTCTCCCAGAACCCAGTAATAAGTGG |
| | | GCTCCTCCCTGGCCTGGACCCCCGTGGTAACCCTATAAGGCGAGGCAGC |
| | | TGCTGTCTGAGGCAGGGAGGGGCTGGTGTGGGAGGCTAAGGGCAGCTGC |
| | | TAAGTTTAGGGTGGCTCCTTCTCTCTTCTTAGAGACAACAGGTGGCTGG |
| | | GGCCTCAGTGCCCAGAAAAGAAAATGTCTTAGAGGTATCGGCATGGGCC |
| | | TGGAGGAGGGGGGACAGGGCAGGGGGAGGCATCTTCCTCAGGACATCGG |
| | | GTCCTAGAGGGAGCGGCCCTGCATGCGAAGATCTTCGAACAAGGCTGTG |
| | | GGGGACTGAGGGCAGGCTGTAACAGGCTTGGGGGCCAGGGCTTATACGT |
| | | GCCTGGGACTCCCAAAGTATTACTGTTCCATGTTCCCGGCGAAGGGCCA |
| | | GCTGTCCCCCGCCAGCTAGACTCAGCACTTAGTTTAGGAACCAGTGAGC |
| | | AAGTCAGCCCTTGGGGCAGCCCATACAAGGCCATGGGGCTGGGCAAGCT |
| | | GCACGCCTGGGTCCGGGGTGGGCACGGTGCCCGGGCAACGAGCTGAAAG |
| | | CTCATCTGCTCTCAGGGGCCCCTCCCTGGGGACAGCCCCTCCTGGCTAG |
| | | TCACACCCTGTAGGCTCCTCTATATAACCCAGGGGCACAGGGGCTGCCC |
| | | TCATTCTACCACCACCTCCACAGCACAGACAGACACTCAGGAGCCAGCC |
| | | AGCGTCGAGATCTTGCTACCAGTGGAACAGCCACTAAGGATTCTGCAGT |
| | | GAGAGCAGAGGGCCAGCTAAGTGGTACTCTCCCAGAGACTGTCTGACTC |
| | | ACGCCACCCCCTCCACCTTGGACACAGGACGCTGTGGTTTCTGAGCCAG |
| | | GTACAGTGACTCCTTTCGGTAAGTGCAGTGGAAGCTGTACACTGCCCAG |
| | | GCAAAGCGTCCGGGCAGCGTAGGCGGGCGACTCAGATCCCAGCCAGTGG |
| | | ACTTAGCCCCTGTTTGCTCCTCCGATAACTGGGGTGACCTTGGTTAATA |
| | | TTCACCAGCAGCCTCCCCCGTTGCCCCTCTGGATCCACTGCTTAAATAC |
| | | GGACGAGGACAGGGCCCTGTCTCCTCAGCTTCAGGCACCACCACTGACC |
| | | TGGGACAGT |
| | | Bold is Synthetic ApoE Enhancer |
| | | Underlined is Mus022 Enhancer |
| | | Italics is CK-hAAT (ΔATG) Promoter |
| CK promoter + | SEQ ID NO: | CCCTGCATGCGAAGATCTTCGAACAAGGCTGTGGGGGACTGAGGGCAGG |
| hAAT(ΔATG) | 11 | CTGTAACAGGCTTGGGGGCCAGGGCTTATACGTGCCTGGGACTCCCAAA |
| | | GTATTACTGTTCCATGTTCCCGGCGAAGGGCCAGCTGTCCCCCGCCAGC |
| | | TAGACTCAGCACTTAGTTTAGGAACCAGTGAGCAAGTCAGCCCTTGGGG |
| | | CAGCCCATACAAGGCCATGGGGCTGGGCAAGCTGCACGCCTGGGTCCGG |
| | | GGTGGGCACGGTGCCCGGGCAACGAGCTGAAAGCTCATCTGCTCTCAGG |
| | | GGCCCCTCCCTGGGGACAGCCCCTCCTGGCTAGTCACACCCTGTAGGCT |
| | | CCTCTATATAACCCAGGGGCACAGGGGCTGCCCTCATTCTACCACCACC |
| | | TCCACAGCACAGACAGACACTCAGGAGCCAGCCAGCGTCGA [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | Italics is CK-hAAT (ΔATG) Promoter |
| | | Italics and no underlining is CK Promoter portion |
| | | Double underlined is hAAT (ΔATG) Promoter portion |
| Mus007 | SEQ ID NO: | AACCATTGTGGGCAGCCAGCCTGGCGGGAGAGGCTAGGGCACGAAGCGC |
| | 12 | CGCCCAGTTACCATGGTAACACTCCCTCGTCCCAACCCCGTCCCAGTTT |
| | | ACTGGCCCAAGGTAAGGCCGGGAGCCGTACTCCAGGGCGACTGGGAGGA |
| | | TCCACTCCCTGCCGCGGCTCTCCCTCCAGTGGACTGCCTTCCCGCCTGC |
| | | CTGGCGGTGGGAGGGCGCGGCAGAGGGACGTCACATCCGGGCGGGTTGG |
| | | TGAGTTCCGGTATTTCAGGGCGTAGCAGGCGGAAGTAAGGGTGAGAGGA |
| | | GGCTGCAACGCCGAGCGGAGGAGGCAGGAACCGGAGCGCGAGCAGTAGC |
| | | TGGGTGGG |
| Mus011 | SEQ ID NO: | CTCCCCTCTTCCTCTTCTTTTAAAACATCCATAGAGCTGCCCAGAATGA |
| | 13 | GTGGAAAGTTTCAGGGTCAGGGAGAACCTCTTCCCTCCCCCACTTTCCA |
| | | TTGTCTAAAATCAGCCTCAGCTGTTGCTAGATATTTTTTTTGCTGGTGA |
| | | TGTCAGTTCATCAAAGCGTCTCACCCTGTGTTCCCCCAGGAGAACAATG |
| | | ATGGGGTGGAGGGAGCTGGAAAGACCATATTAGGAACAAAAGGTGGAGC |
| | | TATGAGCACAGATAAAGACTCAAGTCTGGGGACCTCCTGGTCACTCAGG |
| | | CAGCAGCCCCTTCTTTCTTGCCCCAGTCTCCAGTTCTCCAGTGTTCACA |
| | | GGT |
| Mus035 | SEQ ID NO: | GGCACTGGGGTGTGTCTCCTTAGGACACCCCTGCCCATTACTCTCTCCT |
| | 14 | TCTCCAAATGGGGAGCATGGCTGGGGCTCCCTAACCTCCTGCTTGCGAG |
| | | GCCTCTCTCTGGCCTCTGAGAGGGTCAGTGTCCTGCCCCAACCCATGAG |
| | | ATGACAGACTATAATAGCCACAGGATTAACATAGCAGGCATTGTCTTTC |
| | | TCTGACTATAGGGTGGGTATTATGTGTTCATCAACCATCCTAAAAATAC |
| | | CCGGTAAACAGGTGCAGCCCCTGTGGCTCCAGTCCCCTGGGATCTGTTG |
| | | GCTTCTGGCTGGAGATGAAGATTAGGGCAGAGGAGA |
| LMTP6 | SEQ ID NO: | aggctcagaggcacacaggagtttctgggctcaccctgcccccttccaa |
| | 15 | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggccactacgggtttaggctgccc |
| | | atgtaaggaggcaaggcctggggacacccgagatgcctggttataatta |
| | | acccagacatgtggctgccccccccccccccaacacctgctgcctctaa |
| | | aaataaccctgtccctggtggatcccactacgggtttaggctgcccatg |
| | | taaggaggcaaggcctggggacacccgagatgcctggttataattaacc |
| | | cagacatgtggctgccccccccccccccaacacctgctgcctctaaaaa |
| | | taaccctgtccctggtggatcccactacgggtttaggctgcccatgtaa |
| | | ggaggcaaggcctggggacacccgagatgcctggttataattaacccag |
| | | acatgtggctgccccccccccccccaacacctgctgcctctaaaaataa |
| | | ccctgtccctggtggatcccctgcatgcgaagatcttcgaacaaggctg |
| | | tgggggactgagggcaggctgtaacaggcttgggggccagggcttatac |
| | | gtgcctgggactcccaaagtattactgttccatgttcccggcgaagggc |
| | | cagctgtcccccgccagctagactcagcacttagtttaggaaccagtga |
| | | gcaagtcagcccttggggcagcccatacaaggccatggggctgggcaag |
| | | ctgcacgcctgggtccggggtgggcacggtgcccgggcaacgagctgaa |
| | | agctcatctgctctcaggggcccctccctggggacagcccctcctggct |
| | | agtcacaccctgtaggctcctctatataacccaggggcacaggggctgc |
| | | cctcattctaccaccacctccacagcacagacagacactcaggagccag |
| | | ccagcgtcgagatcttgctaccagtggaacagccactaaggattctgca |
| | | gtgagagcagagggccagctaagtggtactctcccagagactgtctgac |
| | | tcacgccaccccctccaccttggacacaggacgctgtggtttctgagcc |
| | | aggtacagtgactcctttcggtaagtgcagtggaagctgtacactgccc |
| | | aggcaaagcgtccgggcagcgtaggcgggcgactcagatcccagccagt |
| | | ggacttagcccctgtttgctcctccgataactggggtgaccttggttaa |
| | | tattcaccagcagcctcccccgttgcccctctggatccactgcttaaat |
| | | acggacgaggacagggccctgtctcctcagcttcaggcaccaccactga |
| | | cctgggacagt |
| Alpha-Mic/Bik | SEQ ID NO: | aggttaatttttaaaaagcagtcaaaagtccaagtggcccttggcagca |
| Enhancer | 17 | tttactctctctgtttgctctggttaataatctcaggagcacaaacatt |
| (Mic/BikE) | | cc |
| Tandem (2) | SEQ ID NO: | aggttaatttttaaaaagcagtcaaaagtccaagtggcccttggcagca |
| alpha-Mic/Bik | 18 | tttactctctctgtttgctctggttaataatctcaggagcacaaacatt |
| Enhancers | | ccaggttaatttttaaaaagcagtcaaaagtccaagtggcccttggcag |
| (2 Mic/BikE) | | catttactctctctgtttgctctggttaataatctcaggagcacaaaca |
| | | ttcc |
| ApoE Hepatic | SEQ ID NO: | aggctcagaggcacacaggagtttctgggctcaccctgcccccttccaa |
| Control Region | 19 | cccctcagttcccatcctccagcagctgtttgtgtgctgcctctgaagt |
| containing | | ccacactgaacaaacttcagcctactcatgtccctaaaatgggcaaaca |
| ApoE | | ttgcaagcagcaaacagcaaacacacagccctccctgcctgctgacctt |
| Enhancer | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggg |
| Tandem (2) | SEQ ID NO: | aggctcagaggcacacaggagtttctgggctcaccctgcccccttccaa |
| ApoE | 20 | cccctcagttcccatcctccagcagctgtttgtgtgctgcctctgaagt |
| Enhancers | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagaaggctcagaggcacac |
| | | aggagtttctgggctcaccctgcccccttccaacccctcagttcccatc |
| | | ctccagcagctgtttgtgtgctgcctctgaagtccacactgaacaaact |
| | | tcagcctactcatgtccctaaaatgggcaaacattgcaagcagcaaaca |
| | | gcaaacacacagccctccctgcctgctgaccttggagctggggcagagg |
| | | tcagagacctctctg |
| TBG Promoter | SEQ ID NO: | gggctggaagctacctttgacatcatttcctctgcgaatgcatgtataa |
| | 21 | tttctacagaacctattagaaaggatcacccagcctctgcttttgtaca |
| | | actttcccttaaaaaactgccaattccactgctgtttggcccaatagtg |
| | | agaactttttcctgctgcctcttggtgcttttgcctatggcccctattc |
| | | tgcctgctgaagacactcttgccagcatggacttaaacccctccagctc |
| | | tgacaatcctctttctcttttgttttacatgaagggtctggcagccaaa |
| | | gcaatcactcaaagttcaaaccttatcattttttgctttgttcctcttg |
| | | gccttggttttgtacatcagctttgaaaataccatcccagggttaatgc |
| | | tggggttaatttataactaagagtgctctagttttgcaatacaggacat |
| | | gctataaaaatggaaagat |
| ApoE.hAAT | SEQ ID NO: | aggctcagaggcacacaggagtttctgggctcaccctgcccccttccaa |
| | 22 | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggggtacccggggatcttgctacca |
| | | gtggaacagccactaaggattctgcagtgagagcagagggccagctaag |
| | | tggtactctcccagagactgtctgactcacgccaccccctccaccttgg |
| | | acacaggacgctgtggtttctgagccaggtacaatgactcctttcggta |
| | | agtgcagtggaagctgtacactgcccaggcaaagcgtccgggcagcgta |
| | | ggcgggcgactcagatcccagccagtggacttagcccctgtttgctcct |
| | | ccgataactggggtgaccttggttaatattcaccagcagcctcccccgt |
| | | tgcccctctggatccactgcttaaatacggacgaggacagggccctgtc |
| | | tcctcagcttcaggcaccaccactgacctgggacagt |
| Mck Enhancer | SEQ ID NO: | ccactacgggtttaggctgcccatgtaaggaggcaaggcctggggacac |
| (MckE) | 23 | ccgagatgcctggttataattaacccagacatgtggctgcccccccccc |
| | | ccccaacacctgctgcctctaaaaataaccctgtccctggtggatc |
| Tandem (2) | SEQ ID NO: | ccactacgggtttaggctgcccatgtaaggaggcaaggcctggggacac |
| Mck | 24 | ccgagatgcctggttataattaacccagacatgtggctgcccccccccc |
| Enhancers | | ccccaacacctgctgcctctaaaaataaccctgtccctggtggatccca |
| (2 MckE) | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatc |
| Tandem Mck | SEQ ID NO: | ccactacgggtttaggctgcccatgtaaggaggcaaggcctggggacac |
| (3) Enhancers | 25 | ccgagatgcctggttataattaacccagacatgtggctgcccccccccc |
| (3 MckE) | | ccccaacacctgctgcctctaaaaataaccctgtccctggtggatccca |
| | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatcccacta |
| | | cgggtttaggctgcccatgtaaggaggcaaggcctggggacacccgaga |
| | | tgcctggttataattaacccagacatgtggctgccccccccccccccaa |
| | | cacctgctgcctctaaaaataaccctgtccctggtggatc |
| Myosin heavy | SEQ ID NO: | cccttcagattaaaaataactgaggtaagggcctgggtaggggaggtgg |
| chain enhancer | 26 | tgtgagacgctcctgtctctcctctatctgcccatcggccctttgggga |
| (MhcE) | | ggaggaatgtgcccaaggactaaaaaaaggccatggagccagaggggcg |
| | | agggcaacagacctttcatgggcaaaccttggggccctgctgaagcttt |
| | | ggc |
| CK8 Promoter | SEQ ID NO: | ccctgcatgcgaagatcttcgaacaaggctgtgggggactgagggcagg |
| | 27 | ctgtaacaggcttgggggccagggcttatacgtgcctgggactcccaaa |
| | | gtattactgttccatgttcccggcgaagggccagctgtcccccgccagc |
| | | tagactcagcacttagtttaggaaccagtgagcaagtcagcccttgggg |
| | | cagcccatacaaggccatggggctgggcaagctgcacgcctgggtccgg |
| | | ggtgggcacggtgcccgggcaacgagctgaaagctcatctgctctcagg |
| | | ggcccctccctggggacagcccctcctggctagtcacaccctgtaggct |
| | | cctctatataacccaggggcacaggggctgccctcattctaccaccacc |
| | | tccacagcacagacagacactcaggagccagccagcgtcga |
| SPc5.12 | SEQ ID NO: | ggccgtccgccctcggcaccatcctcacgacacccaaatatggcgacgg |
| | 28 | gtgaggaatggtggggagttatttttagagcggtgaggaaggtgggcag |
| | | gcagcaggtgttggcgctctaaaaataactcccgggagttatttttaga |
| | | gcggaggaatggtggacacccaaatatggcgacggttcctcacccgtcg |
| | | ccatatttgggtgtccgccctcggccggggccgcattcctgggggccgg |
| | | gcggtgctcccgcccgcctcgataaaaggctccggggccggcggcggcc |
| | | cacgagctacccggaggagcgggaggcgccaagc |
| Minimal | SEQ ID NO: | gaatggtggacacccaaatatggcgacggttcctcacccgtcgccatat |
| SPc5.12 | 29 | ttgggtgtccgccctcggccggggccgcattcctgggggccgggcggtg |
| | | ctcccgcccgcctcgataaaaggctccggggccggcggcggcccacgag |
| | | ctacccggaggagcgggaggcgccaag |

### 6.2.1 Enhancers

The present inventors have surprisingly discovered multiple enhancers are amenable to tandem positioning while operably linked to one or more promoters. These enhancers when arranged in tandem and operably linked to promoters, including tandem promoters, and a transgene promote tissue specific expression of the transgene.

Accordingly, provided are ApoE enhancers, particularly an ApoE Hepatic Control Region containing an ApoE Enhancer, as in SEQ ID NO: 1 or synthetic ApoE enhancer, as in SEQ ID NO: 7. Also provided are muscle specific enhancers, such as, Mus022, as in SEQ ID NO: 8.

Other enhancers are well known to the skilled person in the art.

### 6.2.2 Promoters

Another aspect of the present invention relates to nucleic acid expression cassettes comprising chimeric regulatory elements designed to confer or enhance liver-specific and muscle-specific expression (including skeletal or muscle specific expression). The invention involves engineering regulatory elements in tandem, including promoter elements, enhancer elements, and optionally introns. Examples include but are not limited to hAAT promoters (SEQ ID NO: 3 and 4) and CK promoter (SEQ ID NO: 9).

The unique combinations of promoter and enhancer sequences provided herein improve transgene expression while maintaining tissue specificity. The novel regulatory element nucleic acids were generated using a method to improve transgene expression from tandem promoters (i.e. two promoter sequences driving expression of the same transgene) by depleting the 3′ promoter sequence of potential ‘ATG’ initiation sites. This approach was employed to improve transgene expression from tandem tissue-specific promoter cassettes (such as those targeting the liver and muscle) as well as promoter cassettes to achieve dual expression in two separate tissue populations (such as liver and skeletal muscle). Ultimately, these designs aim to improve the therapeutic efficacy of gene transfer by providing more robust levels of transgene expression, improved stability/persistence, and induction of immune tolerance to the transgene product. In certain aspects the hAAT promoter with the start codon deleted (ΔATG) is used in an expression cassette provided herein. In certain aspects the CK promoter with the start codon deleted (ΔATG) is used in an expression cassette provided herein.

The CAG promoter (SEQ ID NO: 17) refers to a chimeric promoter constructed from the following sequences: the cytomegalovirus (CMV) early enhancer element (C), the chicken beta-actin promoter (the first exon and the first intron of chicken beta-actin gene) (A), and the splice acceptor of the rabbit beta-globin gene (G). The CAG promoter is frequently utilized in the art to drive high levels of expression in mammalian cells, and is non-preferential with respect to tissue specificity, therefore is typically utilized as a universal promoter.

### 6.2.3 Introns

Another aspect of the present invention relates to nucleic acid expression cassettes comprising an intron within the regulatory cassette. In some embodiments, the intron nucleic acid is a chimeric intron derived from human β-globin and Ig heavy chain (also known as β-globin splice donor/immunoglobulin heavy chain splice acceptor intron, or β-globin/IgG chimeric intron, Reed, R., et al. Genes and Development, 1989). Use of an intron may further induce efficient splicing in eukaryotic cells. Although use of an intron may not indicate increases in expression to an already strong promoter, the presence of an intron may increase the expression level of transgene and can also increase the duration of expression in vivo.

In some embodiments, the intron is a VH4 intron. The VH4 intron nucleic acid can comprise SEQ ID NO: 12 as shown in Table 2 below. The VH4 intron 5′ of the coding sequence may enhance proper splicing and, thus, transgene expression. Accordingly, in some embodiments, an intron is coupled to the 5′ end of a transgene sequence. In other embodiments, the intron is less than 100 nucleotides in length.

TABLE 2
Nucleotide sequences for different introns

| | SEQ | |
|---|---|---|
| Structure | ID | Sequence |
| Chimeric | 30 | GTAAGTATCAAGGTTACAAGACAG |
| intron | | GTTTAAGGAGACCAATAGAAACTG |
| (β-globin/ | | GGCTTGTCGAGACAGAGAAGACTC |
| Ig | | TTGCGTTTCTGATAGGCACCTATT |
| Intron) | | GGTCTTACTGACATCCACTTTGCC |
| | | TTTCTCTCCACAG |
| VH4 intron | 31 | GTGAGTATCTCAGGGATCCAGACA |
| | | TGGGGATATGGGAGGTGCCTCTGA |
| | | TCCCAGGGCTCACTGTGGGTCTCT |
| | | CTGTTCACAG |
| SV40 intron | 32 | GTAAGTTTAGTCTTTTTGTCTTTT |
| | | ATTTCAGGTCCCGGATCCGGTGGT |
| | | GGTGCAAATCAAAGAACTGCTCCT |
| | | CAGTGGATGTTGCCTTTACTTCTA |
| | | G |

In other embodiments, the intron is a chimeric intron derived from human β-globin and Ig heavy chain (also known as β-globin splice donor/immunoglobulin heavy chain splice acceptor intron, or β-globin/IgG chimeric intron) (Table 2, SEQ ID NO: 30). Other introns well known to the skilled person may be employed, such as the chicken β-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron (Table 2, SEQ ID NO: 32).

Other introns well known to the skilled person may be employed.

### 6.2.4 Other Regulatory Elements

6.2.4.1 polyA

Another aspect of the present disclosure relates to expression cassettes comprising a polyadenylation (poly A) site downstream of the coding region of the transgene. Any poly A site that signals termination of transcription and directs the synthesis of a poly A tail is suitable for use in AAV vectors of the present disclosure. Exemplary poly A signals are derived from, but not limited to, the following: the SV40 late gene, the rabbit β-globin gene (SEQ ID NO: 36), the bovine growth hormone (BPH) gene, the human growth hormone (hGH) gene, the synthetic poly A (SPA) site, and the bovine growth hormone (bGH) gene. See, e.g., Powell and Rivera-Soto, 2015, Discov. Med., 19 (102): 49-57. In one embodiment, the polyA signal comprises SEQ ID NO: 33 as shown in Table 3.

TABLE 3
Nucleotide Sequence of the PolyA Signal

| | SEQ ID | |
|---|---|---|
| Structure | NO: | Sequence |
| β-globin | SEQ ID | ataaaggaaatttattttcattg |
| Poly A | | caatagtgtgttggaattttttg |
| signal | NO: 33 | tgtctctca |
| Rabbit | SEQ ID | gatctttttccctctgccaaaaa |
| β-globin | NO: 34 | ttatggggacatcatgaagcccc |
| poly A | | ttgagcatctgacttctggctaa |
| | | taaaggaaatttattttcattgc |
| | | aatagtgtgttggaattttttgt |
| | | gtctctcactcg |

### 6.3. Vectors for Gene Delivery

Another aspect of the present invention relates to the genetic engineering of tandem nucleic acid regulatory elements and incorporating these nucleic acid sequences in a vector expression system. In one embodiment, the vector is a viral vector, including but not limited to recombinant adeno-associated viral (rAAV) vectors (e.g. Gao G., et al 2003 Proc. Natl. Acad. Sci. U.S.A. 100(10):6081-6086), lentiviral vectors (e.g. Matrai, J, et al. 2011, Hepatology 53, 1696-707), retroviral vectors (e.g. Axelrod, J H, et al. 1990. Proc Natl Acad Sci USA; 87, 5173-7), adenoviral vectors (e.g. Brown et al., 2004 Blood 103, 804-10), herpes-simplex viral vectors (Marconi, P. et al. Proc Natl Acad Sci USA. 1996 93(21): 11319-11320; Baez, M V, et al. Chapter 19-Using Herpes Simplex Virus Type 1-Based Amplicon Vectors for Neuroscience Research and Gene Therapy of Neurologic Diseases, Ed.: Robert T. Gerlai, Molecular-Genetic and Statistical Techniques for Behavioral and Neural Research, Academic Press, 2018: Pages 445-477), and retrotransposon-based vector systems (e.g. Soifer, 2004, Current Gene Therapy 4(4):373-384). In another embodiment, the vector is a non-viral vector. rAAV vectors have limited packaging capacity of the vector particles (i.e. approximately 4.7 kb), constraining the size of the transgene expression cassette to obtain functional vectors (Jiang et al., 2006 Blood. 108:107-15). The length of the transgene and the length of the regulatory nucleic acid sequences comprising tandem enhancer(s) and promoter(s) are taken into consideration when selecting a regulatory region suitable for a particular transgene and target tissue.

Another aspect of the present invention relates to a viral vector comprising an expression cassette comprising a nucleic acid regulatory element LMTP24, operably linked to a transgene, in embodiments flanked by ITR sequences. In some embodiments, the expression cassette comprises a nucleic acid regulatory element comprising the nucleic acid sequence of SEQ ID NO: 10, or a sequence that is 99%, 95%, 90%, 85% or 80% identical to SEQ ID NO: 10 and enhances expression of the transgene in liver and skeletal muscle (with, in embodiments, minimal or reduced expression in cardiac tissue).

In another aspect, the expression cassettes are suitable for packaging in an AAV capsid, as such the cassette comprises (1) AAV inverted terminal repeats (ITRs) flank the expression cassette; (2) regulatory control elements, a) promoter/enhancers, such as LMTP24 in Table 1, b) a poly A signal, and c) optionally an intron, in embodiments situated between the promoter and transgene coding sequence; and (3) a transgene providing (e.g., coding for) one or more RNA or protein products of interest. In certain embodiments, the transgene is from Tables 4A-4D.

In embodiments for expressing an intact or substantially intact mAb, the constructs described herein comprise the following components: (1) AAV2 inverted terminal repeats that flank the expression cassette; (2) regulatory control elements, a) promoter/enhancers, such as LMTP24 (SEQ ID NO: 10), b) a poly A signal, and c) optionally an intron; and (3) nucleic acid sequences coding for the heavy chain Fab of an anti-AB (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/250) 3), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651,), anti-ALK1 (e.g., ascrinvacumab), anti-C5 (e.g., tesidolumab, ravulizumab, and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof; and the light chain of an anti-Aβ (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651,), anti-ALK1 (e.g., ascrinvacumab), anti-C5 (e.g., tesidolumab, ravulizumab, and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); wherein the heavy chain (Fab and Fc region) and the light chain are separated by a self-cleaving furin (F)/F2A or furin (F)/T2A or flexible linker, ensuring expression of equal amounts of the heavy and the light chain polypeptides.

In the various embodiments, the target tissue may be neural tissue, bone, kidney, liver, muscle, heart spleen, lung or endothelial tissue, or a particular receptor or tumor, and the regulatory agent is derived from a heterologous protein or domain that specifically recognizes and/or binds that tissue, particularly liver and muscle. The transgenes expressed in liver and muscle are considered systemic expression, since enhanced delivery of liver-expressed or muscle-expressed protein may be sufficient to cross into other tissues including crossing the blood brain barrier to the CNS and delivering therapeutics for treating neurological disorders or neurological symptoms of a systemic disorder.

### 6.3.1 AAV

Another aspect of the present invention relates to expression cassettes suitable for packaging in an AAV capsid, as such the cassette comprises (1) AAV inverted terminal repeats (ITRs) flank the expression cassette; (2) regulatory control elements, consisting essentially of one or more enhancers and one or more promoters, particularly one of the muscle-liver specific regulatory elements provided herein, including LMTP24 (SEQ ID NO: 10), d) a poly A signal, and e) optionally, an intron; and (3) a transgene providing (e.g., coding for) one or more RNA or protein products of interest.

The provided nucleic acids and methods are suitable for use in the production of any isolated recombinant AAV particles, in the production of a composition comprising any isolated recombinant AAV particles, or in the method for treating a disease or disorder in a subject in need thereof comprising the administration of any isolated recombinant AAV particles. As such, the rAAV may be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1. AAV.hu37, AAV.Anc80, AAV. Anc80L65, AAV.7m8, AAV.PHP.B. AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV. Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV. Anc80, AAV. Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12 (6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAV-PHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25:450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29 (9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689, (see, e.g., SEQ ID NOs: 5-38 in '689 publication) WO2009/104964 (see, e.g., SEQ ID NOs: 1-5, 7, 9, 20, 22, 24 and 31 in '964 publication), WO2010/127097 (see, e.g., SEQ ID NOs: 5-38 in '097 publication), and WO 2015/191508 (see, e.g., SEQ ID NOs: 80-294 in '508 publication), and U.S. Appl. Publ. No. 20150023924 (see, e.g., SEQ ID NOs: 1, 5-10 in '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689 (see, e.g., SEQ ID NOs: 5-38 in '689 publication) WO2009/104964 (see, e.g., SEQ ID NOs: 1-5, 7, 9, 20, 22, 24 and 31 in '964 publication), WO 2010/127097 (see, e.g., SEQ ID NOs: 5-38 in '097 publication), and WO 2015/191508 (see, e.g., SEQ ID NOs: 80-294 of in '508 publication), and U.S. Appl. Publ. No. 20150023924 (see, e.g., SEQ ID NOs: 1, 5-10 in '924 publication).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9458517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for used in the production of recombinant AAV encoding a transgene. In some embodiments, provided herein are rAAV viral vectors encoding an anti-VEGF Fab. In some embodiments, provided herein are rAAV8-based viral vectors encoding an anti-VEGF Fab. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9)-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP. In some embodiments, provided herein are rAAV viral vectors encoding anti-kallikrein (anti-pKal) antibody. In some embodiments, provided herein are rAAV8-based or rAAV9)-based viral vectors encoding a pKal antibody Fab or full-length antibody.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV. Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, rAAV particles comprise a capsid protein from an AAV capsid serotype selected from AAV-8 or AAV-9. In some embodiments, the rAAV particles have an AAV capsid serotype of AAV-1 or a derivative, modification, or pseudotype thereof. In some embodiments, the rAAV particles have an AAV capsid serotype of AAV-4 or a derivative, modification, or pseudotype thereof. In some embodiments, the rAAV particles have an AAV capsid serotype of AAV-5 or a derivative, modification, or pseudotype thereof. In some embodiments, the rAAV particles have an AAV capsid serotype of AAV-8 or a derivative, modification, or pseudotype thereof. In some embodiments, the rAAV particles have an AAV capsid serotype of AAV-9 or a derivative, modification, or pseudotype thereof.

In some embodiments, rAAV particles comprise a capsid protein that is a derivative, modification, or pseudotype of AAV-8 or AAV-9 capsid protein. In some embodiments, rAAV particles comprise a capsid protein that has an AAV-8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV-8 capsid protein.

In some embodiments, rAAV particles comprise a capsid protein that is a derivative, modification, or pseudotype of AAV-9 capsid protein. In some embodiments, rAAV particles in the clarified feed comprise a capsid protein that has an AAV-8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV-9 capsid protein.

In additional embodiments, rAAV particles comprise a mosaic capsid. Mosaic AAV particles are composed of a mixture of viral capsid proteins from different serotypes of AAV. In some embodiments, rAAV particles comprise a mosaic capsid containing capsid proteins of a serotype selected from AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10), AAV.rh20, AAV.rh39, AAV.Rh74, AAV.RHM4-1, AAV.hu37, AAV. Anc80, AAV. Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles comprise a mosaic capsid containing capsid proteins of a serotype selected from AAV-1, AAV-2, AAV-5, AAV-6, AAV-7, AAV-8, AAV-9, AAV-10, AAVrh.8, and AAVrh.10. In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV-1, AAV-3, AAV-4, AAV-5, AAV-6, AAV-7, AAV-8, AAV-9, AAV-10 AAV-11, AAV-12, AAV-13, AAV-14, AAV-15 and AAV-16). In additional embodiments, rAAV particles comprise a pseudotyped rAAV particle comprised of a capsid protein of an AAV serotype selected from AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV. Anc80L65, AAV.7m8, AAV.PHP.B. AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In additional embodiments, rAAV particles comprise a pseudotyped rAAV particle containing AAV-8 capsid protein. In additional embodiments, rAAV particles comprise a pseudotyped rAAV particle is comprised of AAV-9) capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In further embodiments, the capsid protein is a chimeric of 2 or more AAV capsid proteins from AAV serotypes selected from AAV1, AAV2, rAAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV21YF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV-8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV-8 capsid protein and one or more AAV capsid proteins from an AAV serotype selected from AAV1, AAV2, AAV5, AAV6, AAV7, AAV9, AAV10, AAVrh.8, and AAVrh.10.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AA6, AAV7, AAV8, AAV9, AAVrh.8, and AAVrh.10.

Methods of Making rAAV Vectors

Another aspect of the present invention involves making molecules disclosed herein. In some embodiments, a molecule according to the invention is made by providing a nucleotide comprising the nucleic acid sequence encoding an AAV capsid protein; and using a packaging cell system to prepare corresponding rAAV particles with capsid coats made up of the capsid protein. In some embodiments, the nucleic acid sequence encodes a sequence having at least 60%, 70%, 80%, 85%, 90%, or 95%, preferably 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, or 95%, preferably 96%, 97%, 98%, 99% or 99.9%, identity to a particular sequence of the AAV capsid protein, while retaining (or substantially retaining) biological function of the AAV capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In certain embodiments, the cap and rep genes are provided by a packaging cell and not present in the viral genome.

In some embodiments, the nucleic acid encoding the capsid protein is cloned into an AAV Rep-Cap helper plasmid in place of the existing capsid gene. When introduced together into host cells, this plasmid helps package an rAAV genome into the capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In preferred embodiments, the rAAVs provide transgene delivery vectors that can be used in therapeutic and prophylactic applications, as discussed in more detail below. The rAAV vector also includes the regulatory control elements discussed supra to influence the expression of the RNA and/or protein products encoded by nucleic acids (transgenes) within target cells of the subject.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV capsid protein.

The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12: S18-S27, which is incorporated by reference herein in its entirety.

The rAAV vector for delivering the transgene to target tissues, cells, or organs, may also have a tropism for that particular target tissue, cell, or organ, e.g. liver and/or muscle, in conjunction with the use of tissue-specific promoters as described herein. The construct can further include additional expression control elements such as introns that enhance expression of the transgene (e.g., introns such as the chicken β-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin poly A signal, human growth hormone (hGH) polyA signal, SV40) late polyA signal, synthetic polyA (SPA) signal, and bovine growth hormone (bGH) poly A signal. See, e.g., Powell and Rivera-Soto, 2015, Discov. Med., 19 (102): 49-57.

In certain embodiments, nucleic acids sequences disclosed herein may be codon-optimized, for example, via any codon-optimization technique known to one of skill in the art (see. e.g., review by Quax et al., 2015, Mol Cell 59:149-161).

In a certain embodiment, the constructs described herein comprise the following components (LMTP24): (1) AAV inverted terminal repeats (ITRs) that flank the expression cassette; (2) control elements, which include a) synthetic ApoE enhancer region (SEQ ID NO: 7), b) Mus022 muscle-specific enhancer (SEQ ID NO: 8), c) CK promoter (SEQ ID NO: 9), (d) human AAT promoter (ΔATG) (SEQ ID NO: 4), d) a poly A signal, and e) optionally an intron; (3) transgene providing (e.g., coding for) one or more RNA or protein products of interest, such as those in Tables 4A-4D.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMANR analysis. Virions may be recovered, for example, by CsCl2 sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or muscle or other cell types may be used, for example, but not limited, to HuH-7. HEK293, fibrosarcoma HT-1080, HKB-11, C2C12 myoblasts, and CAP cells. Once expressed, characteristics of the expressed product (transgene product) can also be determined, including serum half-life, functional activity of the protein (e.g. enzymatic activity or binding to a target), determination of the glycosylation and tyrosine sulfation patterns, and other assays known in the art for determining protein characteristics.

Provided are methods of manufacturing a recombinant AAV comprising culturing a host cell capable of producing a recombinant AAV described herein under conditions appropriate for production of the recombinant AAV comprising an artificial genome with an expression cassette comprising a synthetic promoter operably linked to a transgene. In particular, the method provides (1) culturing a host cell containing (i) an artificial genome comprising AAV ITRs flanking a recombinant cis expression cassette which comprises a nucleic acid regulatory element comprising a composite nucleic acid regulatory element as disclosed herein operably linked to a transgene; (ii) a trans expression cassette lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and (iii) sufficient adenovirus helper functions to permit replication and packaging of the artificial genome by the AAV capsid protein; and (2) recovering recombinant AAV encapsidating the artificial genome from the cell culture. Also provided are host cells containing (i) an artificial genome comprising AAV ITRs flanking a recombinant cis expression cassette which comprises a composite nucleic acid regulatory element disclosed herein operably linked to a transgene; (ii) a trans expression cassette lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and, optionally. (iii) sufficient adenovirus helper functions to permit replication and packaging of the artificial genome by the AAV capsid protein In particular embodiments, the composite nucleic acid regulatory element is LMTP24 or SEQ ID NO: 10. In certain embodiments, the artificial genome comprises a transgene encoding one of the therapeutics listed in Tables 4A-4D.

### 6.4. Therapeutic and Prophylactic Uses

Another aspect relates to therapies which involve administering a transgene operably linked to a composite regulatory element described herein via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

In embodiments, the transgene comprises cDNA that restores protein function to a subject having a genetic mutation(s) in the corresponding native gene. In embodiments, the transgene encodes a therapeutic protein, including therapeutic antibodies or antigen binding fragments and forms thereof, or other protein product with therapeutic effect. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 4A-4D below provides a list of transgenes that may be used in expression cassettes where the transgene is operably linked to a composite regulatory element described herein in an rAAV vector described herein, to treat or prevent the disease with which the transgene is associated, also listed in Tables 4A-4D. In embodiments, the composite regulatory element is LMTP24 (SEQ ID NO: 10) and promotes expression of the transgene in skeletal muscle and liver tissue and, in embodiments, reduced or no detectable expression in cardiac tissue.

TABLE 4A

| | | Possible |
|---|---|---|
| | | AAV |
| | | serotype for |
| | | delivery of |
| Disease | Transgene | transgene |
| MPS I | alpha-L-iduronidase (IDUA) | AAV9 |
| MPS II (Hunter | iduronate-2-sulfatase (IDS) | AAV9 |
| Syndrome) | | |
| ceroid lipofuscinosis | (CLN1, CLN2, CLN10, CLN13), | AAV9 |
| (Batten disease) | a soluble lysosomal protein (CLN5), | |
| | a protein in the secretory pathway | |
| | (CLN11), two cytoplasmic proteins | |
| | that also peripherally associate with | |
| | membranes (CLN4, CLN14), and | |
| | many transmembrane proteins with | |
| | different subcellular locations | |
| | (CLN3, CLN6, CLN7, CLN8, | |
| | CLN12) | |
| MPS IIIa (Sanfilippo | heparan sulfate sulfatase (also called | AAV9, |
| type A Syndrome) | N-sulfoglucosamine sulfohydrolase | Rh10 |
| | (SGSH)) | |
| MPS IIIB (Sanfilippo | N-acetyl-alpha-D-glucosaminidase | AAV9 |
| type B Syndrome) | (NAGLU) | |
| MPS VI (Maroteaux- | arylsulfatase B | AAV8 |
| Lamy Syndrome) | | |
| MPS IVA (Morquio | GALNS | AAV8 |
| syndrome type A) | | |
| MPS IVA (Morquio | GLB1 | AAV8 |
| syndrome type B) | | |
| Osteogenesis | COL1Al and/or COL1A2 | AAV8 |
| Imperfecta Type I, II, | | |
| III, or IV | | |
| Osteogenesis | IFITM5 | AAV8 |
| Imperfecta Type V | | |
| Osteogenesis | SERPINF1 | AAV8 |
| Imperfecta Type VI | | |
| Osteogenesis | CRTAP | AAV8 |
| Imperfecta Type VII | | |
| Osteogenesis | LEPRE1 and/or P3H1 | AAV8 |
| Imperfecta Type VIII | | |
| Osteogenesis | PPIB | AAV8 |
| Imperfecta Type IX | | |
| Gaucher disease (type | Glucocerebrosidase, GBA1 | AAV9 |
| 1, II and III) | | |
| Parkinson’s Disease | Glucocerebrosidase; GBA1 | AAV9 |
| Parkinson’s Disease | dopamine decarboxylase | AAV2 |
| Pompe | acid maltase; GAA | AAV9 |
| Metachromatic | Aryl sulfatase A | Rh10 |
| leukodystrophy | | |
| MPS VII (Sly | beta-glucuronidase | |
| syndrome) | | |
| MPS VIII | glucosamine-6-sulfate sulfatase | |
| MPS IX | Hyaluronidase | |
| Niemann-Pick disease | Sphingomyelinase | |
| Niemann-Pick disease | a npc1 gene encoding a | |
| without | cholesterol metabolizing enzyme | |
| sphingomyelinase | | |
| deficiency | | |
| Tay-Sachs disease | Alpha subunit of beta- | |
| Sandhoff disease | hexosaminidase both alpha | |
| | and beta subunit of beta- | |
| | hexosaminidase | |
| Fabry Disease | alpha-galactosidase | |
| Fucosidosis | Fucosidase (FUCAl gene) | |
| Alpha-mannosidosis | alpha-mannosidase | |
| Beta-mannosidosis | Beta-mannosidase | |
| Wolman disease | cholesterol ester hydrolase | |
| Parkinson’s disease | Neurturin | |
| Parkinson’s disease | glial derived growth factor (GDGF) | |
| Parkinson’s disease | tyrosine hydroxylase | |
| Parkinson’s disease | glutamic acid decarboxylase. | |
| | fibroblast growth factor-2 (FGF-2) | |
| | brain derived growth factor (BDGF) | |
| No disease listed | neuraminidase deficiency with | |
| (Galactosialidosis | betagalactosidase deficiency | |
| (Goldberg syndrome)) | | |
| Spinal Muscular | SMN | AAV9 |
| Atrophy (SMA) | | |
| Friedreich's ataxia | Frataxin | AAV9 |
| | | PHP.B |
| Amyotrophic lateral | SOD1 | Rh10 |
| sclerosis (ALS) | | |
| Glycogen Storage | Glucose-6-phosphatase | AAV8 |
| Disease 1a | | |
| XLMTM | MTM1 | AAV8 or |
| | | AAV9 |
| Crigler Najjar | UGT1A1 | AAV8 |
| CPVT | CASQ2 | AAV9 |
| Rett syndrome | MECP2 | AAV9 |
| Achromatopsia | CNGB3, CNGA3, GNAT2, PDE6C | AAV8 |
| Choroidermia | CDM | AAV8 |
| Danon Disease | LAMP2 | AAV9 |

TABLE 4B

| | | Possible AAV |
|---|---|---|
| | | serotype for |
| | | delivery of |
| Disease | Transgene | transgene |
| Cystic Fibrosis | CFTR | AAV2 |
| Duchenne Muscular Dystrophy | Mini-/Micro-Dystrophin Gene | AAV2, |
| | | AAV8, AAV9 |
| Limb Girdle Muscular Dystrophy Type | human-alpha-sarcoglycan | AAV1 |
| 2C\|Gamma-sarcoglycanopathy | | |
| Advanced Heart Failure | SERCA2a | AAV6 |
| Rheumatoid Arthritis | TNFR:Fc Fusion Gene | AAV2 |
| Leber Congenital Amaurosis | GAA | AAV1 |
| Limb Girdle Muscular Dystrophy Type | gamma-sarcoglycan | AAV1 |
| 2C\|Gamma-sarcoglycanopathy | | |
| Retinitis Pigmentosa | hMERTK | AAV2 |
| Age-Related Macular Degeneration | SFLT01 | AAV2 |
| Becker Muscular Dystrophy and | huFollistatin344 | AAV1 |
| Sporadic Inclusion Body Myositis | | |
| Parkinson's Disease | GDNF | AAV2 |
| Metachromatic Leukodystrophy (MLD) | cuARSA | AAVrh.10 |
| Hepatitis C | anti-HCV shRNA | AAV8 |
| Limb Girdle Muscular Dystrophy Type | hSGCA | AAVrh74 |
| 2D | | |
| Human Immunodeficiency Virus | PG9DP | AAV1 |
| Infections; HIV Infections (HIV-1) | | |
| Acute Intermittant Porphyria | PBGD | AAV5 |
| Leber's Hereditary Optical Neuropathy | PIND4v2 | AAV2 |
| Alpha-1 Antitrypsin Deficiency | alpha1AT | AAVrh10 |
| Pompe Disease | hGAA | AAV9 |
| X-linked Retinoschisis | RS1 | AAV8 |
| Choroideremia | hCHM | AAV2 |
| Giant Axonal Neuropathy | JeT-GAN | AAV9 |
| Duchenne Muscular Dystrophy | micro-Dystrophin | AAVrh74 |
| X-linked Retinoschisis | hRS1 | AAV2 |
| Squamous Cell Head and Neck Cancer; | hAQP1 | AAV2 |
| Radiation Induced Xerostomia | | |
| Hemophilia B | Factor IX | AAVrh10/ |
| | | Rh74 |
| Homozygous FH | hLDLR | AAV8 |
| Dysferlinopathies | rAAVrh74.MHCK7.DYSF.DV | AAVrh74 |
| Hemophilia B | AAV6 ZFP nuclease | AAV6 |
| MPS I | AAV6 ZFP nuclease | AAV6 |
| Rheumatoid Arthritis | NF-KB.IFN-β | AAV5 |
| Batten/CLN6 | CLN6 | AAV9 |
| Sanfilippo Disease Type A | hSGSH | AAV9 |
| Osteoarthritis | 5IL-1Ra | AAV2.5 |
| Achromatopsia | CNGA3 | AAV2tYF |
| Achromatopsia | CNGB3 | AAV8 |
| Ornithine Transcarbamylase (OTC) | OTC | SCAAV8 |
| Deficiency | | |
| Hemophilia A | Factor VIII | LK03/AAV3B |
| Mucopolysaccharidosis II | ZFP nuclease | AAV6 |
| Hemophilia A | ZFP nuclease | AAV6 |
| Wet AMD | anti-VEGF | AAV8 |
| X-Linked Retinitis Pigmentosa | RPGR | AAV2 |
| Mucopolysaccharidosis Type VI | hARSB | AAV8 |
| Leber Hereditary Optic Neuropathy | ND4 | AAV2 |
| X-Linked Myotubular Myopathy | MTM1 | AAV8 |
| Crigler-Najjar Syndrome | UGT1A1 | AAV8 |
| Achromatopsia | CNGB3 | AAV8 |
| Retinitis Pigmentosa | hPDE6B | AAV5 |
| X-Linked Retinitis Pigmentosa | RPGR | AAV2tYF |
| Mucopolysaccharidosis Type 3 B | hNAGLU | AAV9 |
| Duchenne Muscular Dystrophy | GALGT2 | AAVrh74 |
| Arthritis, Rheumatoid; Arthritis, | TNFR:Fc Fusion Gene | AAV2 |
| Psoriatic; Ankylosing Spondylitis | | |
| Idiopathic Parkinson's Disease | Neurturin | AAV2 |
| Alzheimer's Disease | NGF | AAV2 |
| Human Immunodeficiency Virus | tgAAC09 | AAV2 |
| Infections; HIV Infections (HIV-1) | | |
| Familial Lipoprotein Lipase Deficiency | LPL | AAV1 |
| Idiopathic Parkinson's Disease | Neurturin | AAV2 |
| Alpha-1 Antitrypsin Deficiency | hAAT | AAV1 |
| Leber Congenital Amaurosis (LCA) 2 | hRPE65v2 | AAV2 |
| Batten Disease; Late Infantile | CLN2 | AAVrh.10 |
| Neuronal Lipofuscinosis | | |
| Parkinson's Disease | GAD | AAV2 |
| Sanfilippo Disease Type A/ | N-sulfoglucosamine | AAVrh.10 |
| Mucopolysaccharidosis Type IIIA | sulfohydrolase (SGSH) gene | |
| Congestive Heart Failure | SERC2a | AAV1 |
| Becker Muscular Dystrophy and | rAAV1.CMV.huFollistatin344 | AAV1 |
| Sporadic Inclusion Body Myositis | | |
| Parkinson's Disease | hAADC-2 | AAV2 |
| Choroideremia | REP1 | AAV2 |
| CEA Specific AAV-DC-CTL | CEA | AAV2 |
| Treatment in Stage IV Gastric Cancer | | |
| Gastric Cancer | MUC1-peptide-DC-CTL | |
| Leber's Hereditary Optical Neuropathy | scAAV2-PIND4v2 | scAAV2 |
| Aromatic Amino Acid Decarboxylase | hAADC | AAV2 |
| Deficiency | | |
| Hemophilia B | Factor IX | AAVrh10 |
| Parkinson's Disease | AADC | AAV2 |
| Leber Hereditary Optic Neuropathy | Genetic: GS010\|Drug: Placebo | AAV2 |
| SMA-Spinal Muscular Atrophy\|Gene | SMN | AAV9 |
| Therapy | | |
| Hemophilia A | B-Domain Deleted Factor VIII | AAV8 |
| MPS I | IDUA | AAV9 |
| MPS II | IDS | AAV9 |
| CLN3-Related Neuronal Ceroid- | CLN3 | AAV9 |
| Lipofuscinosis (Batten) | | |
| Limb-Girdle Muscular Dystrophy, | hSGCB | rh74 |
| Type 2E | | |
| Alzheimer Disease | APOE2 | rh10 |
| Retinitis Pigmentosa | hMERKTK | AAV2 |
| Retinitis Pigmentosa | RLBP1 | AAV8 |
| Wet AMD | Anti-VEGF antibody | AAV2.7m8 |

TABLE 4C

| ANTIGENS | | ANTIBODIES | INDICATIONS |
|---|---|---|---|
| Nervous | Amyloid beta | solanezumab | Alzheimer’s Disease |
| System | (Aβ or Abeta) | GSK933776 | |
| Targets | peptides | lecanemab | |
| | derived from | | |
| | APP | | |
| | Sortilin | AL-001 | Frontotemporal |
| | | | dementia (FTD) |
| | Tau protein | ABBV-8E12 | Alzheimer’s, |
| | | UCB-0107 | Progressive |
| | | NI-105 | supranuclear palsy, |
| | | (BIIB076) | frontotemporal |
| | | | demential, |
| | | | chronic traumatic |
| | | | encephalopathy, Pick’s |
| | | | complex, primary |
| | | | age-related taupathy |
| | Semaphorin- | VX15/2503 | Huntington’s disease, |
| | 4D | | juvenile Huntington’s |
| | (SEMA4D) | | disease |
| | alpha- | prasinezumab | Parkinson’s disease, |
| | synuclein | NI-202 | synucleinopathies |
| | | (BIIB054) | |
| | | MED-1341 | |
| | superoxide | NI-204 | ALS, Alzheimer’s |
| | dismutase-1 | | Disease |
| | (SOD-1) | | |
| | CGRP | eptinezumab, | Migraines, Cluster |
| | Receptor | fremanezumab | headaches |
| | | galcanezumab | |
| Repulsive guidance | | elezanumab | multiple sclerosis |
| molecule-A | | | |
| Transthyretin (TTR) | | NI-301 | amyloidosis |
| | | PRX-004 | |
| Connective tissue growth | | pamrevlumab | fibrotic diseases, e.g. |
| factor (CTGF) | | | diabetic nephropathy, |
| | | | liver fibrosis, idiopathic |
| | | | pulmonary fibrosis |
| Neuromyelitis | interleukin | Satralizumab | NMO, DR, DME, |
| optica | receptor 6 | Sarilumab | uveitis |
| (NMO)/ | (IL6R) | Tocilizumab | |
| Uveitis | Interleukin 6 | siltuximab, | NMO, DR, DME, |
| targets | | clazakizumab | uveitis |
| | | sirukumab | |
| | | olokizumab | |
| | | gerilimzumab | |
| | CD19 | inebilizumab | NMO |

| | | |
|---|---|---|
| Integrin beta 7 | etrolizumab | ulcerative colitis, |
| | | Crohn’s disease |
| Sclerostin | romosozumab | Osteoporosis, abnormal |
| | (EVENITY ®) | bone loss or weakness |
| Complement Component 5 | Crovalimab | Myasthenia Gravis |
| | eculizumab | |
| | tesidolumab | |
| | ravulizumab | |
| Interleukin receptor 6 | Satralizumab | Adverse immune |
| (IL6R) | Sarilumab | responses |
| | Tocilizumab | (e.g. cytokine storm, |
| Interleukin 6 (IL6) | siltuximab, | CAR-T therapy) |
| | clazakizumab | |
| | sirukumab | |
| | olokizumab | |
| | gerilimzumab | |
| Immunoglobin E (IgE) | omolizumab | Asthma, COPD, |
| | | eosinophilic asthma, |
| | | chronic idiopathic |
| | | urticaria |
| Thymic stromal | tezelipumab | Asthma, COPD |
| lymphopoietin (TSLP) | | |
| Interleukin 5 (IL5) | benralizumab | Asthma, COPD |
| Interleukin 5 receptor | reslizumab | Asthma, COPD, |
| (IL5R) | | eosinophilic asthma |
| Interleukin 13 (IL13) | tralokinumab | Atopic dermatitis |
| Interleukin 31 recptor alpha | nemolizumab | Atopic dermatitis |
| (IL31RA) | | |

TABLE 4D

| ANTIGENS | | ANTIBODIES | INDICATIONS |
|---|---|---|---|
| | Amyloid beta | | Alzheimer’s Disease |
| | (Aβ or Abeta) | | |
| | peptides | | |
| Nervous System | Tau protein | anti-TAU | Alzheimer's, Progressive |
| Targets | | | supranuclear palsy, |
| | | | frontotemporal demential, |
| | | | chronic traumatic |
| | | | encephalopathy, Pick's |
| | | | complex, primary age- |
| | | | related taupathy |
| | CGRP Receptor | erenumab | anti-TAU |
| | | (AIMOVIG ™) | |
| Interleukins or | IL-17A | ixekizumab (TALTZ ®) | Plaque psoriasis, psoriatic |
| interleukin | | secukinumab | arthritis, ankylosing |
| receptors | | (COSENTYX ®) | spondylitis |
| | IL-5 | mepolizumab | Asthma |
| | | (NUCALA ®) | |
| | IL-12/IL-23 | ustekinumab | Psoriasis & Crohn's |
| | | (STELARA ®) | disease |
| | IL-4R | dupilumab | Atopic dermatitis |
| Integrin | | vedolizumab | Ulcerative colitis & |
| | | (ENTYVIO ®) | Crohn's disease |
| | | Natalizumab (anti- | Multiple sclerosis & |
| | | integrin alpha 4) | Crohn's disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab (XGEVA ® | Osteoporosis, increasing |
| | | and | bone mass in breast and |
| | | PROLIA ® | prostate cancer patients, & |
| | | | preventing skeletal-related |
| | | | events due to bone |
| | | | metastasis |
| PD-1, or PD-L1 or PD-L2 | | nivolumab (OPDIVO ®) | Metastatic melanoma, |
| | | pembrolizumab | lymphoma, non-small cell |
| | | (KEYTRUDA ®) | lung carcinoma |
| BLyS (B-lymphocyte stimulator, | | belimumab | Systemic lupus |
| also known as B-cell activating | | (BENLYSTA ®) | erythromatosis |
| factor (BAFF)) | | | |
| Ocular Targets | VEGF | ranibizumab | Wet AMD |
| | | (LUCENTIS ®) | |
| | | bevacizumab | |
| | | (AVASTIN ®) | |
| | | brolucizumab | |
| | Factor D | lampalizumab | Dry AMD |
| | MMP9 | andecaliximab | |
| TNF-alpha | | adalimumab | Rheumatoid arthritis, |
| | | (HUMIRA ®) and | psoriatic arthritis, |
| | | infliximab | ankylosing spondylitis, |
| | | (REMICADE ®) | Crohn's disease, plaque |
| | | | psoriasis, ulcerative colitis |
| Plasma Protein | C5, C5a | Crovalimab | Paroxysmal nocturnal |
| targets | | Eculizumab | hemoglobinuria, atypical |
| | | (SOLIRIS ®) | hemolytic uremic |
| | | tesidolumab | syndrome, complement- |
| | | ravulizumab | mediated thrombotic |
| | | (ULTOMIRIS ®) | microangiopathy |
| | Plasma | Lanadelumab | Hereditary angioedema |
| | kallikrein | (TAKHZYRO ®) | (HAE) |

In one example, a rAAV vector comprising a transgene encoding glial derived neurotrophic factor (GDNF) operably linked to a composite regulatory element described herein finds use in treating/preventing/managing Parkinson's disease. In another example, a rAAV comprising a transgene encoding an anti-kallikrein antibody, such as lanadelumab, operably linked to a composite regulatory element described herein, finds use in treating/preventing/managing hereditary angioedema (HAE). In still another example, a rAAV comprising a transgene encoding a lysosomal enzyme, operably linked to a composite regulatory element described herein, finds use in treating/preventing/managing mucopolysaccharidosis. Generally, the rAAV vector is administered systemically, and following transduction, the vector's production of the protein product is enhanced by an expression cassette employing engineered liver-specific and muscle-specific nucleic acid regulatory elements, including LMTP24 (SEQ ID NO: 10). In embodiments, the expression of the protein product is enhanced compared to expression of the protein product comprising an expression cassette comprising a muscle-specific promoter alone or an expression cassette comprising a liver-specific promoter alone. In embodiments, the expression of the protein product is enhanced compared to expression of the protein product comprising an expression cassette comprising a LMTP6 promoter. For example, the rAAV vector may be provided by intravenous, intramuscular, and/or intra-peritoneal administration.

With respect to the therapeutic antibodies in Tables 4C and 4D, the expression cassettes comprising the regulatory sequences operably linked to the transgene encoding the therapeutic antibody may be packaged in an rAAV for delivery that preferably has an AAV8 capsid, an AAV9) capsid or an AAVrh10 capsid for targeting or expression in liver and/or muscle cells.

In some aspects, the rAAVs of the present invention find use in delivery to target tissues associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject an rAAV where the expression cassette comprises a nucleic acid regulatory element LMTP24 operably linked to a transgene.

Following transduction of target cells, the expression of the protein product is enhanced by employing such liver-specific and muscle-specific expression cassettes. Such enhancement may be measured by the following non-limiting list of determinations such as 1) protein titer by assays known to the skilled person, not limited to sandwich ELISA, Western Blot, histological staining, and liquid chromatography tandem mass spectrometry (LC-MS/MS); 2) protein activity, by assays such as binding assays, functional assays, enzymatic assays and/or substrate detection assays; and/or 3) serum half-life or long-term expression. Enhancement of transgene expression may be determined as efficacious and suitable for human treatment (Hintze, J. P. et al, Biomarker Insights 2011:6 69-78). Assessment of the quantitative and functional properties of a transgene using such in vitro and in vivo cellular, blood and tissue studies have been shown to correlate to the efficacy of certain therapies (Hintze, J. P. et al, 2011, supra), and are utilized to evaluate response to gene therapy treatment of the transgene with the vectors described herein. Comparative assessment may be relative to an rAAV of the same capsid type comprising a expression cassette or recombinant genome that is identical except for the regulatory sequence employed. In an embodiment, the regulatory sequence may be a CAG promoter.

rAAV vectors of the invention also can facilitate delivery, in particular, targeted delivery, of transgenes operably linked to the chimeric regulatory sequences described herein, including but not limited to oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. In some embodiments, the rAAV molecule may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some preferred embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In certain embodiments, the transgene is administered intravenously even if intended to be expressed in the CNS, for example, by forming a depot in the liver where the transgene is expressed and secreted into the bloodstream.

In certain embodiments, the agents of the invention are administered intravenously or intramuscularly and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, the rAAVs can be used for in vivo delivery of transgenes for scientific studies such as gene knock-down with miRNAs, recombinase delivery for conditional gene deletion, gene editing with CRISPRs, and the like.

### 6.5. Pharmaceutical Compositions and Kits

The invention further provides a pharmaceutical composition comprising a pharmaceutically acceptable carrier and an agent of the invention, said agent comprising a rAAV molecule of the invention comprising a transgene cassette wherein the transgene expression is driven by the chimeric regulatory elements described herein. In preferred embodiments, the pharmaceutical composition comprises rAAV combined with a pharmaceutically acceptable carrier for administration to a subject. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soy bean oil, mineral oil, sesame oil and the like. Water is a common carrier when the pharmaceutical composition is administered intravenously or intramuscularly. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In preferred embodiments, the agent of the invention is substantially purified (i.e., substantially free from substances that limit its effect or produce undesired side-effects). In a specific embodiment, the host or subject is an animal, preferably a mammal such as non-primate (e.g., cows, pigs, horses, cats, dogs, rats etc.) and a primate (e.g., monkey such as, a cynomolgus monkey and a human). In a preferred embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 7. EXAMPLES

### 7.1. Example 1-Cis-Regulatory Element (CRE) Discovery for Muscle-Specific Transgene Expression

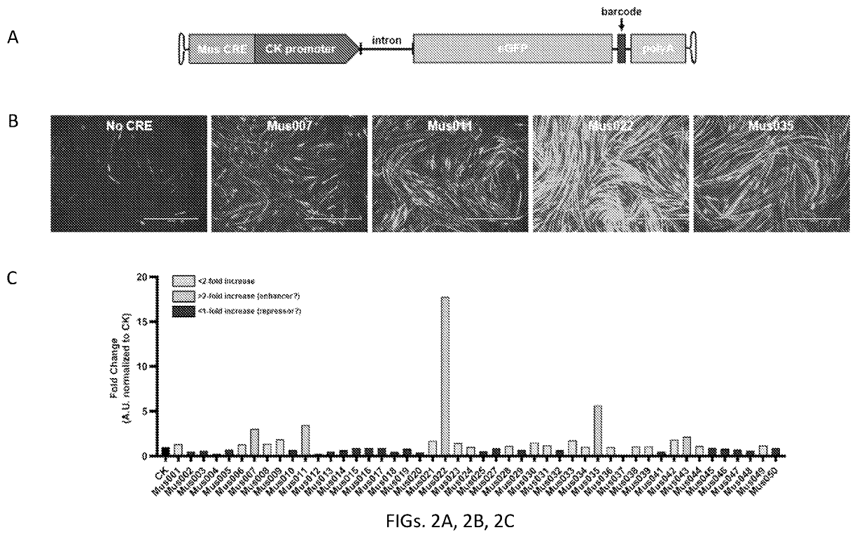

Candidate cis-regulatory element (CRE) sequences derived from regions proximal to genes that are specifically or significantly enriched in skeletal or cardiac muscle were identified in the ENCODE database (Davis, C. et al., 2018 Nucleic Acids Res. The Encyclopedia of DNA elements (ENCODE): data portal update; 46(D1):D794-D801. doi: 10.1093/nar/gk×10⁸¹). Compact sequences were cloned upstream of the muscle-specific CK promoter in cis reporter plasmids individually barcoded between the eGFP coding sequence and RBG polyA tail (FIG. 2A). The selected CRE sequences plus CK promoter were screened for specificity as enhancers by characterizing transgene expression in C2C12 myotubes following individual cis plasmid transfection. Enhancer activity of each CRE was semi-quantitated by calculating the fold-change of transgene expression (GFP fluorescence) normalized to the control plasmid (CK promoter with no upstream CRE) (FIGS. 2B, 2C). Enhancers (CREs) Mus007, Mus011, Mus0) 22 and Mus035 were identified as exhibiting a greater than 2-fold increase normalized to the control plasmid (CK promoter with no upstream CRE), with Mus022 and Mus035 exhibiting significantly increased enhancement of transgene expression.

### 7.2. Example 2-Tandem Liver/Muscle-Specific Promoter Driving Gene Expression of Vectorized Antibody in Non-Human Primates

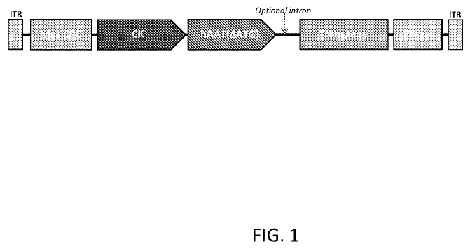

A tandem promoter cassette was engineered to express transgene within both liver and muscle cells. It contains the Mus022 enhancer followed by the complete CK promoter (FIG. 1). Downstream of the CK promoter is the hAAT promoter depleted of ATG sites. Theoretically, the tandem promoter cassette design will allow expression of two transcripts, one specific for muscle and one for liver cell expression. By depleting the ATG site in the second promoter element hAAT, it is anticipated that the first ‘ATG’ initiation codon encountered in both transcripts occurs at the intended site of translation of the gene of interest. This promoter is named LMTP24.

Antibody cDNA-based plasmids were constructed comprising a transgene comprising codon optimized nucleotide sequences encoding the heavy and light chain sequences of an antibody. The nucleotide sequences encoding the light chain and full-length chain were heavy (including Fc) separated by a Furin-T2A linker RKRR(GSG)EGRGSLLTCGDVEENPGP, SEQ ID NO: 35) to create a bicistronic vector expressing a full-length antibody, or a flexible linker to create a single chain Fv (ScFv-Fc) antibody. The cis plasmids additionally included the LMTP24 (SEQ ID NO: 10) or LMTP6 (SEQ ID NO: 15) promoter.

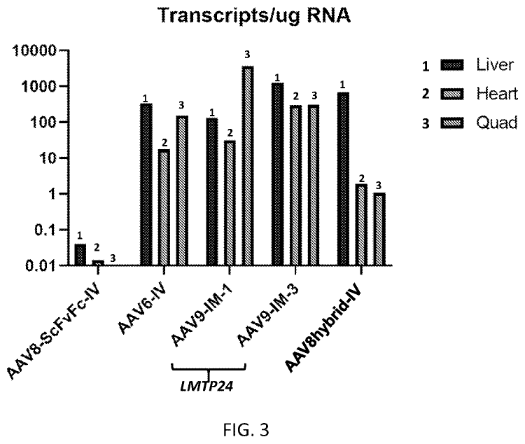

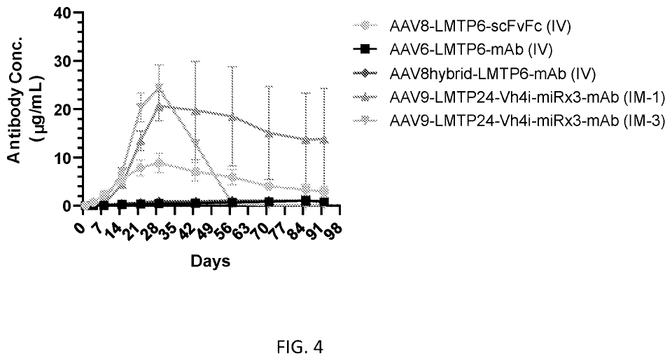

Each genome (cis plasmid) is encapsidated by a different capsid for comparison of expression of vectorized antibody under control of the two tandem promoters. The LTMP24 driven transgenes also included a VH4 intron, and miRNA embedded into the UTR in order to reduce immune response in the animal (possible anti-transgene antibodies). Such genomes were packaged in AAV as outlined in Table 5, then rAAV particles evaluated by administration to cynomolgus monkeys and evaluating biodistribution and potency of the transduction and expression of the transgene in liver, heart and skeletal muscle (quadriceps). All procedures were in compliance with the Animal Welfare Act, the Guide for the Care and Use of Laboratory Animals, and the Office of Laboratory Animal Welfare. Whole blood samples (serum) were collected on days 1, 4, 8, 15, 22, 29, 43, 57, 71, 85, and 92 (necropsy). Animals were anesthetized and sacrificed 92 days post injection, then snap-frozen samples of major peripheral tissues (Liver, Heart, Skeletal Muscle) as well as whole blood samples were collected for vector copy number analysis and gene expression analysis by RNA quantification (FIG. 3). Whole blood was analyzed for antibody concentration in serum (FIG. 4). The mean value of the antibody expression from AAV9-LMTP24 IM injected capsids is about 2-3-fold higher than the AAV8-ScFvFc IV injected capsid.

TABLE 5

| | Dose | Number of | Dose | Dose |
|---|---|---|---|---|
| Test Article | Route | animals | (GC/kg) | Volume |
| AAV8-LMTP6- | IV | 3 | 1E13 | 2 mL/kg |
| ScFvFc mAb | | | | |
| AAV6-LMTP6-IgG | IV | 2 | 1E13 | 2 mL/kg |
| mAb | | | | |
| AAV9-LMTP24- | IMx1 | 3 | 1E13 | 0.2 |
| VH4-IgG | | | | mL/kg/dose |
| mAb(miRNAx1) | | | | site |
| AAV9-LMTP24- | IMx3 | 3 | 1E13 | 0.067 |
| VH4-IgG | | | | mL/kg/dose |
| mAb(miRNAx3) | | | | site |
| AAV8-hybrid- | IV | 1 | 1E13 | 2 mL/kg |
| LMTP6-IgG mAb | | | | |

ScFvFc mAb = single chain Fv-Fc antibody; IgG mAb = full-length antibody; IV = intravenous; IM = intramuscular; IMx1 = one IM injection site; IMx3 = three IM injection sites

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A recombinant expression cassette comprising a composite nucleic acid regulatory element comprising a) Muscle cis regulatory element (CRE) and b) at least two promoters arranged in tandem wherein at least one promoter is a muscle specific promoter and one promoter is hAAT, wherein the hAAT is start-codon deleted (ΔATG) (SEQ ID NO: 4), operably linked to a transgene.

2. A recombinant expression cassette comprising a composite nucleic acid regulatory element comprising a) Muscle cis regulatory element (CRE) and b) a muscle specific promoter, operably linked to a transgene.

3. The recombinant expression cassette of claim 1 or 2 wherein the muscle specific promoter is a CK promoter (SEQ ID NO: 9).

4. The recombinant expression cassette of claim 1 or 2 wherein the muscle specific promoter is from Table 1.

5. The recombinant expression cassette of any one of claims 1 to 4 wherein the muscle CRE is Mus022, Mus077, Mus011 or Mus035.

6. The recombinant expression cassette of claim 5 wherein the muscle CRE is Mus022.

7. The recombinant expression cassette of any one of claims 1 to 6 further comprising one or two copies of synthetic ApoE (SEQ ID NO: 7) either 5′ of or 3′ of the muscle CRE.

8. The recombinant expression cassette of any one of claims 1 to 7, wherein the nucleic acid regulatory element is LMTP24 (SEQ ID NO: 10).

9. The recombinant expression cassette of any one of claims 1 to 8 which further comprises an intron sequence between the nucleic acid regulatory element and the transgene.

10. The expression cassette of any one of claims 1 to 9, where the transgene is a gene or nucleic acid encoding any of the therapeutics listed in Tables 4A-4D.

11. The expression cassette of any one of claims 1 to 10, wherein the transgene encodes a therapeutic antibody, or antigen binding fragment thereof.

12. A vector comprising the expression cassette of any one of claims 1 to 11.

13. The vector of claim 12, further comprising AAV ITRs flanking the expression cassette.

14. The vector of claim 12 or 13 wherein the cassette is suitable for packaging in an AAV capsid.

15. The vector of any one of claims 12 to 14 comprising an artificial genome comprising (1) AAV inverted terminal repeats (ITRs) flanking the expression cassette; (2) an expression cassette comprising (a) a composite nucleic acid regulatory control element comprising a nucleic acid sequence of SEQ ID NO: 10; b) a poly A signal, c) optionally an intron; and d) a transgene coding for one or more RNA or protein products to which the composite nucleic acid regulatory element is operably linked.

16. An rAAV particle comprising the vector of any one of claims 12 to 15, and a capsid protein from an AAV capsid serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

17. A method for enhancing expression of a transgene in a subject, said method comprising delivery of viral vectors comprising a nucleic acid expression cassette having a 5′ to 3′ arrangement of a) a Mus022 sequence, b) at least one muscle-specific promoter and at least one liver-specific promoter, wherein the 3′ promoter comprises a deleted start codon, c) a transgene; and d) a polyadenylation signal sequence.

18. The method of claim 17 wherein the muscle specific promoter is CK promoter (SEQ ID NO: 9) and the liver-specific promoter is an hAAT (ΔATG) promoter.

19. The method of claim 17 or 18 further comprising a synthetic ApoE enhancer either 5′ of or 3′ of the Mus022 sequence.

20. The method of any one of claims 17 to 19 wherein the nucleic acid expression cassette comprises a nucleic acid regulatory element of LMTP24 (SEQ ID NO: 10).

21. The method of any one of claims 17 to 20, wherein the viral vector is administered intravenously or intramuscularly.

22. The method of any one of claims 17 to 21, wherein transgene expression is enhanced in the circulation or systemically compared to a muscle-specific promoter alone or a liver-specific promoter alone.

23. The method of any one of claims 17 to 22, wherein the transgene expression is enhanced in the liver or skeletal muscle.

24. A method of treatment comprising administering rAAVs comprising the expression cassettes of any one of claims 1-11, vectors of any one of claims 12 to 15, or rAAV of claim 17.

25. A pharmaceutical composition for use in treating a disease or disorder in a subject in need thereof comprising recombinant AAV particles comprising an expression cassette having a synthetic ApoE enhancer sequence (SEQ ID NO: 7) and/or a Mus022 enhancer sequence (SEQ ID NO: 8) upstream of a muscle-specific promoter and a liver-specific promoter, wherein at least the muscle specific promoter or the liver-specific promoter comprises a deleted start codon (ΔATG), operably linked to a transgene.

26. The composition of claim 25 wherein the muscle specific promoter is the CK promoter (SEQ ID NO: 9) and the liver-specific promoter is hAAT (SEQ ID NO: 3 or 4).

27. The composition of claim 26 wherein the hAAT promoter is 3′ of the muscle specific promoter and has a deleted start codon.

28. The composition of any one of the claims 17 to 27, wherein the expression cassette comprises a composite regulatory sequence having the nucleic acid sequence of SEQ ID NO: 10.

29. The composition of any one of claims 17 to 28, wherein the transgene is selected from Tables 4A-4D.

30. The composition of any one of claims 17 to 29, wherein the transgene encodes a therapeutic antibody, or antigen binding fragment thereof.

31. The composition of any one of claims 17 to 30, wherein the rAAV is administered intravenously or intramuscularly.

32. A method of producing recombinant AAVs comprising:
(a) culturing a host cell containing:
(i) an artificial genome comprising a cis expression cassette flanked by AAV ITRs, wherein the cis expression cassette comprises a composite nucleic acid regulatory element comprising a) a synthetic ApoE Enhancer and a Mus022 enhancer, and b) at least two promoters arranged in tandem wherein at least one promoter is a CK promoter and at least one promoter is a hAAT, wherein the hAAT is start-codon deleted (ΔATG), operably linked to a transgene coding for one or more RNA or protein products;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
(iii) sufficient adenovirus helper functions to permit replication and packaging of the artificial genome by the AAV capsid protein; and
(b) recovering recombinant AAV encapsidating the artificial genome from the cell culture.

33. The method of claim 32, wherein the composite nucleic acid regulatory element is LMTP24 (SEQ ID NO: 10).

34. A host cell comprising (i) a plasmid comprising a cis expression cassette flanked by AAV ITRs, wherein the cis expression cassette comprises a composite nucleic acid regulatory element comprising a) a synthetic ApoE Enhancer and a Mus022 enhancer, and b) at least two promoters arranged in tandem wherein at least one promoter is a CK promoter and at least one promoter is a hAAT, wherein the hAAT is start-codon deleted (ΔATG), operably linked to a transgene coding for one or more RNA or protein products.

35. The host cell of claim 34, wherein the composite nucleic acid regulatory element is LMTP24 (SEQ ID: NO: 10).
